(19) United States
(12) Patent Application Publication
(10) Pub. No.: US 2022/0315982 A1
(43) Pub. Date: Oct. 6, 2022

(54) METHODS FOR IDENTIFICATION OF ANTIGEN BINDING SPECIFICITY OF ANTIBODIES

(71) Applicant: VANDERBILT UNIVERSITY, Nashhvikke, TN (US)

(72) Inventors: Marion Francis SETLIFF, Nashville, TN (US); Ivelin Stefanov GEORGIEV, Brentwood, TN (US); Andrea SHIAKOLAS, Nashville, TN (US)

(21) Appl. No.: 17/640,475

(22) PCT Filed: Sep. 4, 2020
(86) PCT No.: PCT/US20/49330

§ 371 Date: Mar. 4, 2022

Related U.S. Application Data
(60) Provisional application No. 62/895,687, filed on Sep. 4, 2019.
(60) Provisional application No. 62/913,432, filed on Oct. 10, 2019.

(51) Int. Cl. C12Q 1/6804; C12N 15/10; C12Q 1/70

(52) U.S. Cl. CPC C12Q 1/6804; C12N 15/1096; C12Q 1/70

(57) ABSTRACT

The present disclosure relates to a method for simultaneous detection of antigens and antigen specific antibodies. LIBRA-seq (Linking B Cell Receptor to Antigen specificity through sequencing) is developed to simultaneously recover both antigen specificity and paired heavy and light chain BCR sequence. LIBRA-seq is a next-generation sequencing-based readout for BCR-antigen binding interactions that utilizes oligonucleotides (oligos) conjugated to recombinant antigens.

### CROSS REFERENCE TO RELATED APPLICATIONS

This application claims the benefit of U.S. Provisional Patent Application Ser. No. 62/895,687 filed Sep. 4, 2019 and U.S. Provisional Patent Application Ser. No. 62/913,432 filed Oct. 10, 2019, the disclosures of which are expressly incorporated herein by reference.

### STATEMENT REGARDING FEDERALLY SPONSORED RESEARCH

This invention was made with government support under Grant No. R01 AI131722 awarded by the National Institutes of Health. The government has certain rights in the invention.

### REFERENCE TO SEQUENCE LISTING

The Sequence Listing submitted Sep. 4, 2020, as a text file named “10644_104WO1_Sequence_Listing,” created on Sep. 4, 2020, and having a size of 676342 bytes, is hereby incorporated by reference.

### FIELD

The present disclosure relates to methods for identification of antigen binding signal from a sequencing-based readout and determination of antibody sequence-antigen specificity associations.

### BACKGROUND

The antibody repertoire—the collection of antibodies present in an individual—responds efficiently to invading pathogens due to its exceptional diversity and ability to fine-tune antigen specificity via somatic hypermutation (Briney et al., 2019; Rajewsky, 1996; Soto et al., 2019). This antibody repertoire is a rich source of potential therapeutics, but its size makes it difficult to examine more than a small cross-section of the total repertoire (Brekke and Sandlie, 2003; Georgiou et al., 2014; Wang et al., 2018; Wilson and Andrews, 2012). Historically, a variety of approaches have been developed to characterize antigen-specific B cells in human infection and vaccination samples. The methods most frequently used include single-cell sorting with fluorescent antigen baits (Scheid et al., 2009; Wu et al., 2010), screens of immortalized B cells (Buchacher et al., 1994; Stiegler et al., 2001), and B cell culture (Bonsignori et al., 2018; Huang et al., 2014; Walker et al., 2009, 2011). However, these methods to couple functional screens with sequences of the variable heavy (V_{H}) and variable light (V_{L}) immunoglobulin genes are low throughput; generally, individual B cells can only be screened against a few antigens simultaneously. What is needed are high-throughput systems and methods for the simultaneous detection of antigens and antigen specific antibodies.

### SUMMARY

In some aspects, disclosed herein is a method for simultaneous detection of an antigen and an antibody that specifically binds said antigen, comprising:
labeling a plurality of antigens with unique antigen barcodes;
providing a plurality of barcode-labeled antigens to a population of B-cells; allowing the plurality of barcode-labeled antigens to bind to the population of B-cells;
washing unbound antigens from the population of B-cells;
separating the B-cells into single cell emulsions;
introducing into each single cell emulsion a unique cell barcode-labeled bead;
preparing a single cell cDNA library from the single cell emulsions;
performing PCR amplification reactions to produce a plurality of amplicons, wherein the amplicons comprise: 1) the cell barcode and the antigen barcode, 2) the cell barcode and an antibody sequence, and 3) a unique molecular identifier (UMI);
sequencing the plurality of amplicons;
removing a sequence lacking the cell barcode, the UMI, or the antigen barcode;
aligning the antibody sequence to a reference library of immunoglobulin V, D, J and C sequences;
constructing a UMI count matrix comprising the cell barcode, the antigen barcode, and the antibody sequence;
determining a LIBRA-seq score; and
determining that the antibody specifically binds an antigen if the LIBRA-seq score of the antibody for the antigen is increased in comparison to a control sample.

In some embodiments, the barcode-labeled antigens are labeled with a first barcode comprising a DNA sequence or an RNA sequence. In some embodiments, the cell barcode-labeled beads are labeled with a second barcode comprising a DNA sequence or an RNA sequence.

In some embodiments, the antibody sequence comprises an immunoglobulin heavy chain (VDJ) sequence, or an immunoglobulin light chain (VJ) sequence.

In some embodiments, the barcode-labeled antigens comprise an antigen from a pathogen or an animal In some embodiments, the antigen from a pathogen comprises an antigen from a virus. In some embodiments, the antigen from a virus comprises an antigen from human immunodeficiency virus (HIV), an antigen from influenza virus, or an antigen from respiratory syncytial virus (RSV).

In some embodiments, the method of any preceding aspect further comprises determining a level of somatic hypermutation of the antibody specifically binding to the antigen

In some embodiments, the method of any preceding aspect further comprises determining a length of a complementarity-determining region (CDR) of the antibody specifically binding to the antigen.

In some embodiments, the method of any preceding aspect further comprises determining a motif of a CDR of the antibody specifically binding to the antigen. In some embodiments, the CDR is selected from the group consisting of CDRH1, CDRH2, CDRH3, CDRL1, CDRL2, and CDRL3.

In another aspect, disclosed herein is a method of determining a broadly neutralizing antibody to a pathogen, said method comprising:
labeling a plurality of antigens derived from the pathogen with unique antigen barcodes;
providing a plurality of barcode-labeled antigens to a population of B-cells;
allowing the plurality of barcode-labeled antigens to bind to the population of B-cells;
washing unbound antigens from the population of B-cells;
separating the B-cells into single cell emulsions;
introducing into each single cell emulsion a unique cell barcode-labeled bead;
preparing a single cell cDNA library from the single cell emulsions;
performing PCR amplification reactions to produce a plurality of amplicons, wherein the amplicons comprise: 1) the cell barcode and the antigen barcode, 2) the cell barcode and an antibody sequence, and 3) a unique molecular identifier (UMI); sequencing the plurality of amplicons;
removing a sequence lacking a cell barcode, unique molecular identifier (UMI), or an antigen barcode;
aligning the antibody sequence to a reference library of immunoglobulin V, D, J and C sequences;
constructing a UMI count matrix comprising the cell barcode, the antigen barcode, and the antibody sequence;
determining a LIBRA-seq score; and
determining that the antibody is a broadly neutralizing antibody if the LIBRA-seq scores of the antibody for two or more antigens are increased in comparison to a control.

In some aspects, disclosed herein is a polynucleotide comprising a sequence set forth in the specification.

In some aspects, disclosed herein is a polypeptide, wherein the polypeptide is encoded by a polynucleotide sequence set forth in the specification.

In some aspects, disclosed herein is a polypeptide comprising a sequence set forth in FIG. 2 or FIG. 3.

In some aspects, disclosed herein is a therapeutic antibody comprising the polypeptide of any preceding aspect.

### DESCRIPTION OF DRAWINGS

The accompanying figures, which are incorporated in and constitute a part of this specification, illustrate aspects described below.

FIG. 1. LIBRA-seq assay schematic and validation. (A.) Schematic of LIBRA-seq assay. Fluorescently-labelled, DNA-barcoded antigens are used to sort antigen-positive B cells before co-encapsulation of single B cells with bead-delivered oligos using droplet microfluidics. Bead-delivered oligos index both cellular BCR transcripts and antigen barcodes during reverse transcription, enabling direct mapping of BCR sequence to antigen specificity following sequencing. Note: elements of the depiction are not shown to scale, and the number and placement of oligonucleotides on each antigen can vary. (B.) The assay was initially validated on Ramos B cell lines expressing BCR sequences of known neutralizing antibodies VRC01 and Fe53 with a three-antigen screening library: BG505, CZA97 and H1 A/New Caledonia/20/99. (C.) Between the minimum (y-axis, top) and maximum (y-axis, bottom) LIBRA-seq score for each antigen, the ability of each of 100 cutoffs was tested for its ability to classify each VRC01 cell and FE53 cell as antigen positive or negative, where antigen positive is defined as having a LIBRA-seq score greater than or equal to the cutoff being evaluated and antigen negative is defined as having a LIBRA-seq score below the cutoff. At each cutoff, the percent of total VRC01 cells (left column of each antigen subpanel) and percent of total FE53 (right columns) that are classified as positive is represented on a white (0%) to dark purple (100%) color scale. (D.) The LIBRA-seq score for each pair of antigens for each B cell was plotted. Each axis represents the range of LIBRA-seq scores for each antigen. Density of total cells is shown, with purple to yellow indicating lowest to highest number of cells, respectively. (E.) The LIBRA-seq score for BG505 (y-axis) and CZA97 (x-axis) for each VRC01 B cell was plotted. Each axis represents the range of LIBRA-seq scores for each antigen. Density of total cells is shown, with purple to yellow indicating lowest to highest number of cells, respectively.

FIG. 2. LIBRA-seq applied to a human B cell sample from HIV-infected donor NIAID 45. (A.) LIBRA-seq experiment setup consisted of three antigens in the screening library: BG505, CZA97, and H1 A/New Caledonia/20/99, and the cellular input was donor NIAID45 PBMCs. (B.) After bioinformatic processing and filtering of cells recovered from single-cell sequencing, the LIBRA-seq score for each antigen was plotted (total=866). Each axis represents the range of LIBRA-seq scores for each antigen. Density of total cells is shown, with purple to yellow indicating lowest to highest number of cells, respectively. (C.) 29 VRC01 lineage B cells were identified and examined for phylogenetic relatedness to known lineage members and for sequence features, with phylogenetic tree showing relatedness of previously identified VRC01 lineage members (black) and members newly identified using LIBRA-seq (red). Each row represents an antibody. Sequences were aligned using clustalW and a maximum likelihood tree was inferred using maximum likelihood inference. The resulting tree was visualized using an inferred VRC01 unmutated common ancestor (UCA) (accession MK032222) as the root. For each antibody isolated from LIBRA-seq, a heat map of the LIBRA-seq scores for each antigen (BG505, CZA97, and H1 A/New Caledonia/20/99) is shown; blue-white-red represents low to high scores, respectively. Levels of somatic hypermutation (SHM) at the nucleotide level for the heavy and light chain variable genes as reported by the international ImMunoGeneTics information system (IMGT) are displayed as bars, with the numerical percentage value listed to the right of the bar; length of the bar corresponds to level of SHM Amino acid sequences of the complementarity determining region 3 for the heavy chain (CDRH3) and the light chain (CDRL3) for each antibody are displayed. The tree was visualized and annotated using iTol (Letunic and Bork, 2019). CDRH3 Sequences in FIG. 2C: AMRDYCRDDNCNKWDLRH (SEQ ID NO: 770); AMRDYCRDDNCNRWDLRH (SEQ ID NO: 771); AMRDYCRDDSCNIWDLRH (SEQ ID NO: 917); AMRDYCRDDNCNIWDLRH (SEQ ID NO: 918); VRTAYCERDPCKGWVFPH (SEQ ID NO: 919); VRRFVCDHCSDYTFGH (SEQ ID NO: 920); VRRGHCDHCYEWTLQH (SEQ ID NO: 921); VRRGSCDYCGDFPWQY (SEQ ID NO: 922); VRRGSCGYCGDFPWQY (SEQ ID NO: 923); VRGSSCCGGRRHCNGADCFNWDFQY (SEQ ID NO: 924); VRGRSCCGGRRHCNGADCFNWDFQY (SEQ ID NO: 925); VRGKSCCGGRRYCNGADCFNWDFEH (SEQ ID NO: 926); VRGRSCCDGRRYCNGADCFNWDFEH (SEQ ID NO: 927); TRGKYCTARDYYNWDFEH (SEQ ID NO: 928); TRGKYCTARDYYNWDFEY (SEQ ID NO: 929); TRGKNCDDNWDFEH (SEQ ID NO: 930); TRGKNCNYNWDFEH (SEQ ID NO: 931). CDRL3 sequences in FIG. 2C: QHRET (SEQ ID NO: 907); QFLEN (SEQ ID NO: 906); QDQEF (SEQ ID NO: 904); QDRQS (SEQ ID NO: 905); QQFEF (SEQ ID NO: 908); QCLEA (SEQ ID NO: 903); QSFEG (SEQ ID NO: 915); QCFEG (SEQ ID NO: 902); QQYEF (SEQ ID NO: 911). (D.) Antigen specificity as predicted by LIBRA-seq was validated by ELISA for a subset of monoclonal antibodies belonging to the VRC01 lineage. ELISA data are representative from at least two independent experiments. (E.) Neutralization of Tier 1, Tier 2, and control viruses by VRC01 and newly identified VRC01 lineage members, 2723-3131, 2723-4186, and 2723-3055. (F.) Sequence characteristics and antigen specificity of newly identified antibodies from donor NIAID 45. Percent identity is calculated at the nucleotide level, and CDR length and sequences are noted at the amino acid level. LIBRA-seq scores for each antigen are displayed as a heat map with the overall minimum LIBRA-seq score for each antigen displayed as light yellow, 0 as white, and the overall maximum LIBRA-seq score for each antigen as purple. ELISA binding data against BG505, CZA97, and H1 A/New Caledonia/20/99 is displayed as a heat map of the AUC analysis with AUC of 0 displayed as light yellow, 50% max as white, and maximum AUC as purple. ELISA data are representative from at least two independent experiments. VDJ junction sequences in FIG. 2F: ARHRADYDFWNGNNLRGYFDP (SEQ ID NO: 939); ARHRANYDFWGGSNLRGYFDP (SEQ ID NO: 940); ARHRADYDFWGGSNLRGYFDP (SEQ ID NO: 941); ARDEVLRGSASWFLGPNEVRHYGMDV (SEQ ID NO: 942); VGRQKYISGNVGDFDF (SEQ ID NO: 943); ATGRIAASGFYFQH (SEQ ID NO: 944); AREHTMIFGVAEGFWFDP (SEQ ID NO: 775); VTMSGYHVSNTYLDA (SEQ ID NO: 945); ARGRVYSDY (SEQ ID NO: 946); VJ junction sequences in FIG. 2F: QQYGSSPTT (SEQ ID NO: 912); QQYGTSPTT (SEQ ID NO: 913); MQSLQLRS (SEQ ID NO: 899); QQYTNLPPALN (SEQ ID NO: 914); HHYNSFSHT (SEQ ID NO: 892); SSRDTDDISVI (SEQ ID NO: 916); QQYANSPLT (SEQ ID NO: 910); QQSGTSPPWT (SEQ ID NO: 909). Sequences in FIG. 2 can also be found in Table 3 and Table 4.

FIG. 3. LIBRA-seq applied to a sample from NIAID donor N90. (A.) LIBRA-seq experiment setup consisted of nine antigens in the screening library: 5 HIV-1 Env (KNH1144, BG505, ZM197, ZM106.9, B41), and 4 influenza HA (H1 A/New Caledonia/20/99, H1 A/Michigan/45/2015, H5 Indonesia/5/2005, H7 Anhui/1/2013), and the cellular input was donor N90 PBMCs. (B.) 18 VRC38 lineage B cells were identified and examined for phylogenetic relatedness to known lineage members as well as for sequence features, with phylogenetic tree showing relatedness of previously identified VRC38 lineage members (black) and members newly identified using LIBRA-seq (red). Each row represents an antibody. Sequences were aligned using clustalW and a maximum likelihood tree was inferred using maximum likelihood inference. The resulting tree was visualized using the germline IGHV3-23*01 gene as the root. For each antibody isolated from LIBRA-seq, a heat map of the LIBRA-seq scores for each HIV antigen (BG505, B41, KNH1144, ZM106.9 and ZM197) is shown; blue-white-red represents low to high scores, respectively. Levels of somatic hypermutation (SHM) at the nucleotide level for the heavy and light chain variable genes as reported by IMGT are displayed as bars, with the numerical percentage value listed to the right of the bar; length of the bar corresponds to level of SHM. Amino acid sequences of the complementarity determining region 3 for the heavy chain (CDRH3) and the light chain (CDRL3) for each antibody are displayed. The tree was visualized and annotated using iTol (Letunic and Bork, 2019). CDRH3 sequences in FIG. 3B: VRGPSSGWWYHEYSGLDV (SEQ ID NO: 932); IRGPESGWFYHYYFGLGV (SEQ ID NO: 933); ARGPSSGWHLHYYFGMGL (SEQ ID NO: 934); VRGPSSGWHLHYYFGMDL (SEQ ID NO: 935); VRGASSGWHLHYYFGMDL (SEQ ID NO: 936). CDRL3 sequences in FIG. 3B: MQARQTPRLS (SEQ ID NO: 897); MQSLETPRLS (SEQ ID NO: 937); MQSLQTPRLS (SEQ ID NO: 938); MEALQTPRLT (SEQ ID NO: 894); METLQTPRLT (SEQ ID NO: 896); MESLQTPRLT (SEQ ID NO: 895). (C.) Sequence characteristics and antigen specificity of newly identified antibodies from donor N90. Percent identity is calculated at the nucleotide level, and CDR length and sequences are noted at the amino acid level. LIBRA-seq scores for each antigen are displayed as a heat map with the overall minimum LIBRA-seq score for each antigen displayed as light yellow, 0 as white, and the overall maximum LIBRA-seq score for each antigen as purple and ELISA binding data is displayed as a heat map of the AUC analysis calculated from the data with AUC of 0 displayed as light yellow, 50% max as white, and maximum AUC as purple. ELISA data are representative from at least two independent experiments. VDJ junction sequences in FIG. 3C: ARDAGERGLRGYSVGFFDS (SEQ ID NO: 947); AKVVAGGQLRYFDWQEGHYYGMDV (SEQ ID NO: 948). VJ junction sequences in FIG. 3C: HQYGTTPYT (SEQ ID NO: 893); MQSLQTPHS (SEQ ID NO:900). (D.) Neutralization of Tier 2, and control viruses by newly identified antibody 3602-870. (E.) BG505 DS-SOSIP binding to 3602-870 IgG alone or in presence of PGT145 Fab (green), PGT122 Fab (blue) and VRC01 Fab (black). (F.) For each combination of HIV SOSIPs (left) or influenza hemagglutinins (right), the number of B cells with high LIBRA-seq scores (>=1) is displayed as a bar graph. The combinations of antigens are displayed by filled in dots indicating a given antigen is part of the indicated combination. Each combination is mutually exclusive. The total number of B cells with high LIBRA-seq scores for each antigen is indicated as a horizontal bar on the bottom left of each subpanel. Sequences in FIG. 3 can also be found in Table 5 and Table 6.

FIG. 4. Sequence properties of the antigen-specific B cell repertoire. (A.) V gene usage of broadly HIV-reactive B cells. For each IGHV gene, the number of B cells with high LIBRA-seq scores for 3 or more HIV SOSIP variants is displayed as a bar, including B cells with high scores to any 3, 4 or 5 SOSIPs. (B.) Each dot represents a IGHV germline gene, plotted based on the number of B cells reactive to only 1 SOSIP (x axis) and the number of B cells reactive to 3 or more SOSIPs (y axis) that are assigned to that respective IGHV germline gene. IGHV genes above the dotted line (y=x) could indicate enrichment for broad SOSIP antigen reactivity, and IGHV genes below the dotted line — enrichment for strain-specific SOSIP recognition. (C.) IGHV gene identity (y-axis) is plotted for cells with high (>=1) LIBRA-seq scores for each of 1 through 5 HIV-1 SOSIP antigens (x-axis). Each distribution is displayed as a kernel density estimation, where wider sections of a given distribution represent a higher probability that B cells possess a given germline identity percentage. The median of each distribution is displayed as a white dot, the interquartile range is displayed as a thick bar, and a thin line extends to 1.5× the interquartile range.

FIG. 5. Purification of DNA-barcoded antigens. (A.) After barcoding each antigen with a unique oligonucleotide, antigen-oligo complexes are run on size exclusion chromatography to remove excess, unconjugated oligonucleotide from the reaction mixture. DNA-barcoded BG505 was run on the Superose 6 Increase 10/300 GL column and all other DNA-barcoded antigens were run on the Superdex 200 Increase 10/300 GL on the AKTA FPLC system. For size exclusion chromatography, dotted lines indicate DNA-barcoded antigens and fractions taken. The second peak indicates excess oligonucleotide from the conjugation reaction. (B.) Binding of VRC01 or Fe53 Ramos B-cell lines to DNA-barcoded, fluorescently labeled antigens via flow cytometry. VRC01 cells bound to DNA-barcoded BG505-PE, DNA-barcoded CZA97-PE, and not DNA-barcoded H1 A/New Caledonia/20/99-PE. Fe53 cells bound to DNA-barcoded H1 A/New Caledonia/20/99-PE.

FIG. 6. Ramos B-cell line sorting scheme. (A.) Gating scheme for fluorescence activated cell sorting of Ramos B-cell lines. VRC01 and Fe53 Ramos B cells were mixed in a 1:1 ratio and then stained with LiveDead-V500 and a DNA-barcoded antigen screening library consisting of BG505-PE, CZA97-PE, and H1 A/New Caledonia/20/99-PE. Gates as drawn are based on gates used during the sort, and percentages from the sort are listed. (B.) For each experiment, the categorization of the number of Cellranger-identified (10× Genomics) cells after sequencing is shown. Each category (row) is a subset of cells of the previous category (row).

FIG. 7. Identification of antigen-specific B cells from donor NIAID 45 PBMCs. (A.) Gating scheme for fluorescence activated cell sorting of donor NIAID 45 PBMCs. Cells were stained with LiveDead-V500, CD14-V500, CD3-APCCy7, CD19-BV711, IgG-FITC, and a DNA-barcoded antigen screening library consisting of BG505-PE, CZA97-PE, and H1 A/New Caledonia/20/99-PE. Gates as drawn are based on gates used during the sort, and percentages from the sort are listed. These plots show a starting number of 50,187 total events. Due to the visualization parameters, 18 IgG-positive, antigen-positive cells are displayed, but 3400 IgG were sorted and supplemented with 13,000 antigen positive B cells for single cell sequencing. A small aliquot of donor NIAID45 PBMCs were used for fluorescence minus one (FMO) staining, and were stained with the same antibody panel as listed above with the exception of the HIV-1 and influenza antigens. (B.) LIBRA-seq scores for BG505 (x-axis) and CZA97 (y-axis) are shown. Each axis represents the range of LIBRA-seq scores for each antigen. Density of total cells is shown. Overlaid on the density plot are the 29 VRC01 lineage members (dots) indicated in light green. (C.) Antigen specificity as predicted by LIBRA-seq was validated by ELISA for a variety of antibodies isolated from donor NIAID 45. Antibodies were tested for binding to BG505, CZA97, and H1 A/New Caledonia/20/99. ELISA data are representative from at least two independent experiments.

FIG. 8. Characterization of antibody lineage 2121. (A.) Binding of BG505 DS-SOSIP trimer to (a) PGT145 IgG, (b) VRC01 IgG, (c) 17b IgG, and (d) 2723-2121 IgG. (B.) Inhibition of BG505 DS-SOSIP binding to 2723-2121 IgG in presence of VRC34 Fab (diamond), PGT145 Fab (square) and VRC01 Fab (triangle). (C.) Neutralization of Tier 1, Tier 2, and control viruses by antibody 2723-2121 and VRC01. Results are shown as the concentration of antibody (in □g/ml) needed for 50% inhibition (IC5o). (D.) Levels of ADCP, ADCD, ADCT-PKH26 and ADCC displayed by antibody 2723-2121 compared to VRC01. HIVIG was used as a positive control and the anti-RSV mAb Palivisumab as a negative control.

FIG. 9. Identification of antigen-specific B cells from donor N90 PBMCs. (A.) Gating scheme for fluorescence activated cell sorting of donor N90 PBMCs. Cells were stained LiveDead-APCCy7, CD14-APCCy7, CD3-FITC, CD19-BV711, and IgG-PECy5 with and a DNA-barcoded antigen screening library consisting of BG505-PE, KNH1144-PE, ZM197-PE, ZM106.9-PE, B41-PE, H1 A/New Caledonia/20/99-PE, H1 A/Michigan/45/2015-PE, H5 Indonesia/5/2005-PE, H7 Anhui/1/2013-PE. Gates as drawn are based on gates used during the sort, and percentages from the sort are listed. 5450 IgG positive, antigen positive cells were sorted and supplemented with 1480 IgG negative, antigen positive B cells for single cell sequencing. A small aliquot of donor N90 PBMCs were used for fluorescence minus one (FMO) staining, and were stained with the same antibody panel as listed above without the antigen screening library. (B.) Antigen specificity as predicted by LIBRA-seq was validated by ELISA for two antibodies isolated from donor N90. Antibodies were tested for binding to all antigens from the screening library: 5 HIV-1 SOSIP (BG505, KNH1144, ZM197, ZM106.9, B41), and 4 influenza HA (H1 A/New Caledonia/20/99, H1 A/Michigan/45/2015, H5 Indonesia/5/2005, H7 Anhui/1/2013). ELISA data are representative from at least two independent experiments.

FIG. 10. Each graph shows the LIBRA-seq score for an HIV antigen (y-axes) vs. an influenza antigen (x-axes) in the screening library. The 901 cells that had a LIBRA-seq score above one for at least one antigen are displayed as individual dots. IgG cells (591 of 901) are colored orange and cells of all other isotypes are colored blue. Red lines on each axis indicate a LIBRA-seq score of one. Only 9 of the 591 IgG cells displayed high LIBRA-seq scores for at least one HIV-1 antigen and one influenza antigen, confirming the ability of the technology to successfully discriminate between diverse antigen specificities.

FIG. 11. Sequencing preprocessing and quality statistics. (A.) Quality filtering of the antigen barcode FASTQ files. Fastp (Chen et al., 2018) was used to trim adapters and remove low-quality reads using default parameters. Shown are read and base statistics generated from the output html report from each of the Ramos B cell experiment (left), primary B cell experiment from donor NIAID45 (middle), and primary B cell experiment from donor N90 (right). (B.) Shown is a distribution of insert sizes of the antigen barcode reads from the Ramos B cell line experiment, as output from the fastp html report. (C.) Shown is a distribution of insert sizes of the antigen barcode reads from the donor NIAID45 experiment, as output from the fastp html report. (D.) Shown is a distribution of insert sizes of the antigen barcode reads from the donor NIH90 experiment, as output from the fastp html report.

FIG. 12. Architecture of antigen barcode library. The antigen barcode library is composed of the cell barcode, unique molecular identifier, a capture sequences (the template switch oligo sequence), and an antigen barcode.

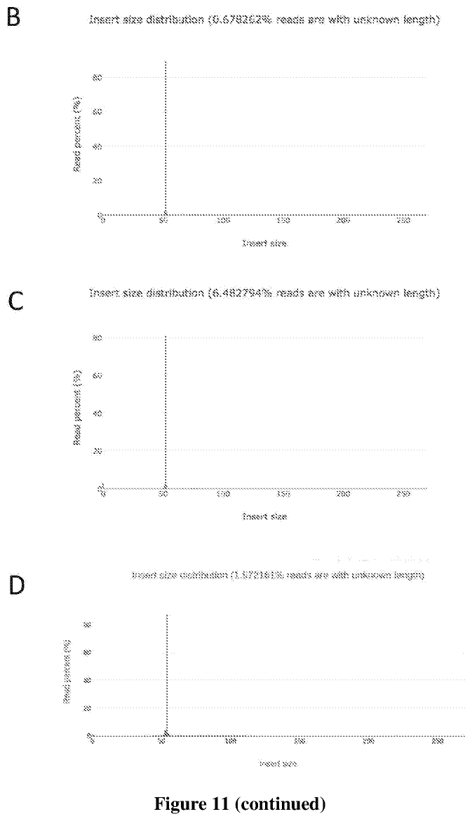

FIG. 13. Schematic of cell barcode — antigen barcode UMI count matrix. This is created from the sequencing of antigen barcode libraries and used in subsequent analysis to determine antigen specificity.

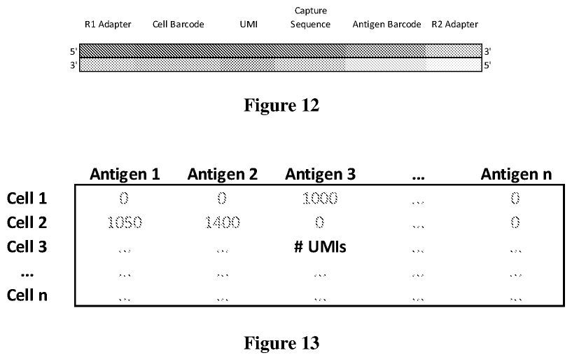

### DETAILED DESCRIPTION

Recent advances in next-generation sequencing (NGS) enable high-throughput interrogation of antibody repertoires at the sequence level, including paired heavy and light chains (Busse et al., 2014; Dekosky et al., 2013; Tan et al., 2014). However, annotation of NGS antibody sequences for their cognate antigen partner(s) generally requires synthesis, production and characterization of individual recombinant monoclonal antibodies (DeFalco et al., 2018; Setliff et al., 2018). Recent efforts to develop new antibody screening technologies have sought to overcome throughput limitations while still uniting antibody sequence and functional information. For example, natively-paired human BCR heavy and light chain amplicons can be expressed and screened as Fab (Wang et at, 2018) or scFV (Adler et al., 2017b, 2017a) in a yeast display system. Although these various antibody discovery technologies have led to the identification of a number of potently neutralizing antibodies, they remain limited by the number of antigens against which single cells can simultaneously be screened efficiently.

LIBRA-seq (LInking B Cell Receptor to Antigen specificity through sequencing) is developed to simultaneously recover both antigen specificity and paired heavy and light chain BCR sequence. LIBRA-seq is a next-generation sequencing-based readout for BCR-antigen binding interactions that utilizes oligonucleotides (oligos) conjugated to recombinant antigens. Antigen barcodes are recovered during paired-chain BCR sequencing experiments and bioinformatically mapped to single cells. The LIBRA-seq method was applied to PBMC samples from two HIV-infected subjects, and from these, HIV- and influenza-specific antibodies were successfully identified, including both known and novel broadly neutralizing antibody (bNAb) lineages. LIBRA-seq is high-throughput, scalable, and applicable to many targets. This single, integrated assay enables the mapping of monoclonal antibody sequences to panels of diverse antigens theoretically unlimited in number and facilitates the rapid identification of cross-reactive antibodies that serves as therapeutics or vaccine templates.

Disclosed herein are systems and methods for simultaneous detection of antigens and antigen specific antibodies.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human

“Nucleotide,” “nucleoside,” “nucleotide residue,” and “nucleoside residue,” as used herein, can mean a deoxyribonucleotide or ribonucleotide residue, or other similar nucleoside analogue. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The term “polynucleotide” refers to a single or double stranded polymer composed of nucleotide monomers.

The method and the system disclosed here including the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner Typically, the disclosed primers hybridize with the disclosed nucleic acids or region of the nucleic acids or they hybridize with the complement of the nucleic acids or complement of a region of the nucleic acids.

The term “amplification” refers to the production of one or more copies of a genetic fragment or target sequence, specifically the “amplicon”. As it refers to the product of an amplification reaction, amplicon is used interchangeably with common laboratory terms, such as “PCR product.”

The term “polypeptide” refers to a compound made up of a single chain of D- or L-amino acids or a mixture of D- and L-amino acids joined by peptide bonds.

As used herein, the term “antigen” refers to a molecule that is capable of stimulating an immune response such as by production of antibodies specific for the antigen. Antigens of the present invention can be, for example, an antigen from human immunodeficiency virus (HIV), an antigen from influenza virus, or an antigen from respiratory syncytial virus (RSV). Antigens of the present invention can also be, for example, a human antigen (e.g. an oncogene-encoded protein).

In the present invention, “specific for” and “specificity” means a condition where one of the molecules involved in selective binding. Accordingly, an antibody that is specific for one antigen selectively binds that antigen and not other antigens.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to specifically interact with the HIV virus, such that the HIV viral infection is prevented, inhibited, reduced, or delayed. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

Each antibody molecule is made up of the protein products of two genes, heavy-chain gene and light-chain gene. The heavy-chain gene is constructed through somatic recombination of V, D, and J gene segments. In humans, there are 51 VH, 27 DH, 6 JH, 9 CH gene segments on human chromosome 14. The light-chain gene is constructed through somatic recombination of V and J gene segments. There are 40 Vκ, 31 Vλ, 5 Jκ, 4 Jλ gene segments on human chromosome 14 (80 VJ). The heavy-chain constant domains that correspond to the different classes of immunoglobulins are called α, δ, ε, γ, and μ, respectively. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)₂, Fab′, Fab, Fv, sFv, scFv and the like, including hybrid fragments. Thus, fragments of the antibodies that retain the ability to bind their specific antigens are provided. For example, fragments of antibodies which maintain HIV virus binding activity are included within the meaning of the term “antibody or antigen binding fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

“Therapeutically effective amount” or “therapeutically effective dose” of a composition refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as coughing relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Methods

In some aspects, disclosed herein is a method for simultaneous detection of an antigen and an antibody that specifically binds said antigen, comprising:
labeling a plurality of antigens with unique antigen barcodes;
providing a plurality of barcode-labeled antigens to a population of B-cells;
allowing the plurality of barcode-labeled antigens to bind to the population of B-cells;
washing unbound antigens from the population of B-cells;
separating the B-cells into single cell emulsions;
introducing into each single cell emulsion a unique cell barcode-labeled bead;
preparing a single cell cDNA library from the single cell emulsions;
performing PCR amplification reactions to produce a plurality of amplicons, wherein the amplicons comprise: 1) the cell barcode and the antigen barcode, 2) the cell barcode and an antibody sequence, and 3) a unique molecular identifier (UMI);
sequencing the plurality of amplicons;
removing a sequence lacking the cell barcode, the UMI, or the antigen barcode;
aligning the antibody sequence to a reference library of immunoglobulin V, D, J and C sequences;
constructing a UMI count matrix comprising the cell barcode, the antigen barcode, and the antibody sequence;
determining a LIBRA-seq score; and
determining that the antibody specifically binds an antigen if the LIBRA-seq score of the antibody for the antigen is increased in comparison to a control sample.

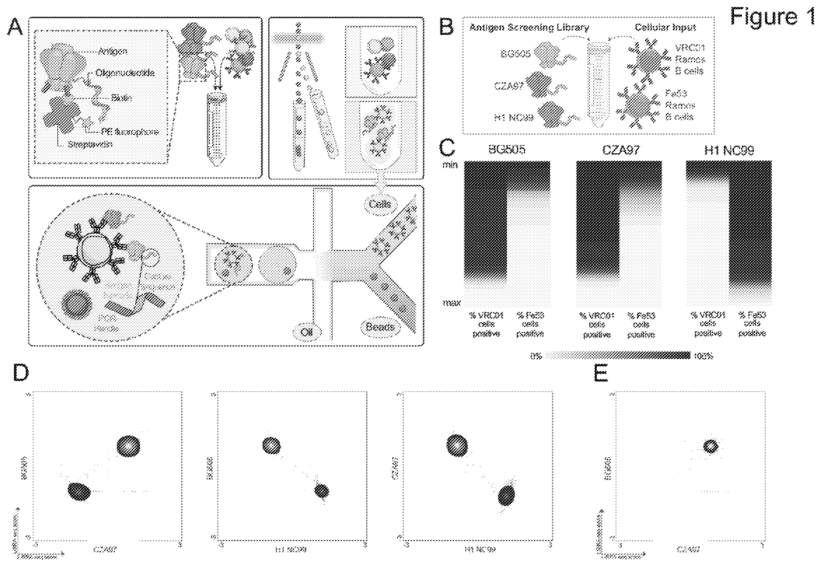

Following a LIBRA-seq experiment, there are 2 resulting pairs of FASTQ files: (1) B cell receptor libraries (containing heavy and light chain contigs), and (2) antigen barcode libraries (containing antigen-identifying DNA barcode sequences from the antigen screening library). In some embodiments, it should be understood that the methods described herein are for uniting the information from these two sequencing libraries. Accordingly, in some embodiments, the above noted step of removing a sequence lacking the cell barcode, the UMI, or the antigen barcode is for removing a sequence from the antigen barcode library lacking the cell barcode, the UMI, or the antigen barcode. The general structure of the antigen barcode should be look like, for example, FIG. 1 disclosed herein. The methods describe here are for processing the antigen barcodes. The processing serves two purposes: (1) quality control and annotation of sequenced reads, and (2) identification of binding signal from the annotated sequenced reads. Before the following steps are carried out, the BCR libraries are processed in order to determine the list of cell barcodes that have a VDJ sequence.

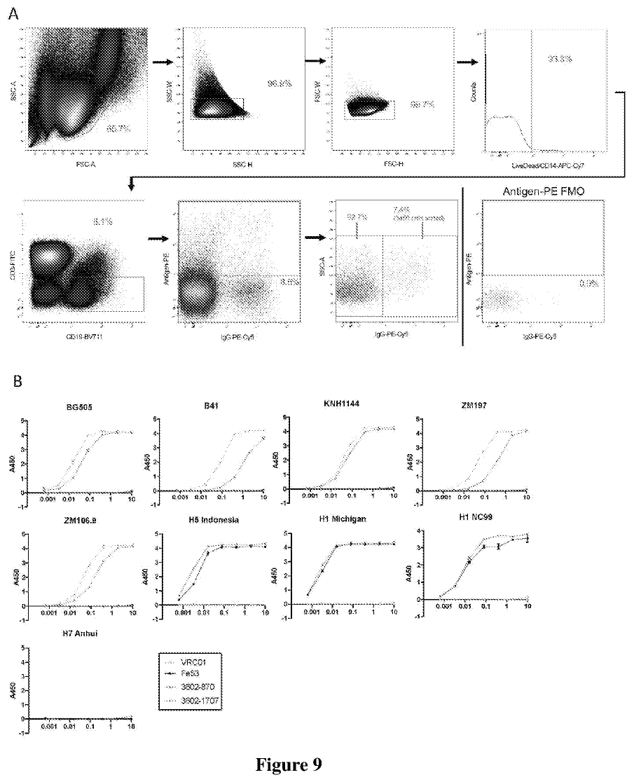

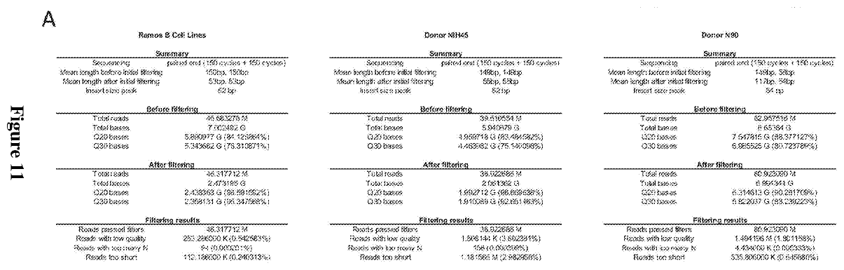

Processing of antigen barcode reads and BCR sequence contigs. A pipeline shown herein takes paired-end fastq files of oligo libraries as input, processes and annotates reads for cell barcode, UMI, and antigen barcode, and generates a cell barcode—antigen barcode UMI count matrix. BCR contigs are processed using cellranger (10× Genomics) using GRCh38 as reference. For the antigen barcode libraries, initial quality and length filtering is carried out by fastp (Chen et al., 2018) using default parameters for filtering. This results in only high-quality reads being retained in the antigen barcode library (FIG. 11). In a histogram of insert lengths, this results in a sharp peak of the expected insert size of 52-54 (FIG. 9B-9C). Fastx_collapser is then used to group identical sequences and convert the output to deduplicated fasta files. Then, having removed low-quality reads, just the R2 sequences were processed, as the entire insert is present in both R1 and R2. Each unique R2 sequence (or R1, or the consensus of R1 and R2) was processed one by one using the following steps:

(1) The reverse complement of the R2 sequence is determined (Skip step 1 if using R1).

(2) The sequence is screened for possessing an exact match to any of the valid 10× cell barcodes present in the filtered_contig.fasta file output by cell ranger during processing of BCR V(D)J fastq files. Sequences without a BCR-associated cell barcode are discarded.

(3) The 10 bases immediate 3′ to the cell barcode are annotated as the read's UMI.

(4) The remainder of the sequence 3′ to the UMI is screened for a 13 or 15 bp sequence with a hamming distance of 0, 1, or 2 to any of the antigen barcodes used in the screening library. Following this processing, only sequences around the expected lengths are retained (the lengths of sequences can be from more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 bases shorter to more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 bases longer than the expected lengths), thus allowing for a deletion, an insertion outside the cell barcode, or bases flanking the cell barcode.

This general process requires that sequences possess all elements needed for analysis (cell barcode, UMI, and antigen barcode), but is permissive to insertions or deletions in the TSO region between the UMI and antigen barcode. After processing each sequence one-by-one, cell barcode—UMI—antigen barcode collisions are screened. Any cell barcode—UMI combination (indicative of a unique oligo molecule) that has multiple antigen barcodes associated with it is removed. A cell barcode—antigen barcode UMI count matrix is then constructed, which served as the basis of subsequent analysis. Additionally, the BCR contigs are aligned (filtered_contigs.fasta file output by Cellranger, 10× Genomics) to IMGT reference genes using HighV-Quest (Alamyar et al., 2012). The output of HighV-Quest is parsed using ChangeO (Gupta et al., 2015), and merged with the UMI count matrix.

The above stated procedure can be summarized as the following steps:

1) Remove low quality reads;

2) Remove reads too long or too short to be a valid antigen barcode read containing a cell barcode, UMI, and antigen barcode;

3) For each quality read, annotate:
a. Cell barcode,
b. UMI
c. Antigen barcode, allowing for sequencing/PCR errors by using a hamming distance threshold.

Determination of LIBRA-seq Score. Starting with the UMI count matrix, all counts of more than one UMIs (for example, more than 1, 2, 3, 4, 5, 6, 7, 8, 9, 10, 11, 12, 13, 14, 15, 16, 17, 18, 19, 20 UMIs) were set to 0, with the idea that these low counts can be attributed to noise. After this, the UMI count matrix was subset to contain only cells with a count of one or more UMIs than the minimum value in the above noted step of noise filtering for at least 1 antigen. The centered-log ratios (CLR) of each antigen UMI count for each cell were then calculated (Mimitou et al., 2019; Stoeckius et al., 2017, 2018). Because UMI counts were on different scales for each antigen, due to differential oligo loading during oligo-antigen conjugation, the CLRs UMI counts were rescaled using the StandardScaler method in scikit learn (Pedregosa and Varoquaux, 2011). Lastly, A correction procedure was performed to the z-score-normalized CLRs from UMI counts of 0, setting them to the minimum for each antigen for donor NIAID 45 and N90 experiments, and to −1 for the Ramos B cell line experiment. These CLR-transformed, Z-score-normalized, corrected values served as the final LIBRA-seq scores. LIBRA-seq scores were visualized using Cytobank (Kotecha et al., 2010).

Identification of sequence feature—antigen specificity associations. Following determination of LIBRA-seq scores (above), and because antibody sequence is united with antigen specificity (in the form of a LIBRA-seq score), sequence-specificity associations can be made.

Accordingly, in some embodiments, the method of any preceding aspect further comprises determining a level of somatic hypermutation of the antibody specifically binding to the antigen

In some embodiments, the method of any preceding aspect further comprises determining a length of a complementarity-determining region (CDR) of the antibody specifically binding to the antigen. The term “complementarity determining region (CDR)” used herein refers to an amino acid sequence of an antibody variable region of a heavy chain or light chain. CDRs are necessary for antigen binding and determine the specificity of an antibody. Each variable region typically has three CDRs identified as CDR1 (CDRH1 or CDRL1, where “H” indicates the heavy chain CDR1 and “L” indicates the light chain CDR1), CDR2 (CDRH2 or CDRL2), and CDR3 (CDRH3 or CDRL3). The CDRs may provide contact residues that play a major role in the binding of antibodies to antigens or epitopes. Four framework regions, which have more highly conserved amino acid sequences than the CDRs, separate the CDR regions in the VH or VL.

Accordingly, in some embodiments, the method of any preceding aspect further comprises determining a motif of a CDR of the antibody specifically binding to the antigen. In some embodiments, the CDR is selected from the group consisting of CDRH1, CDRH2, CDRH3, CDRL1, CDRL2, and CDRL3.

In some embodiments, the method of any preceding aspect further comprises identification of IGHV, IGHD, IGHJ, IGKV, IGKJ, IGLV, or IGLJ genes, or combinations thereof, associated with any particular combination of antigen specificities.

In some embodiments, the method of any preceding aspect further comprises identification of mutations in heavy or light FW1, FW2, FW3 or FW4 associated with any particular combination of antigen specificities.

In some embodiments, the method of any preceding aspect further comprises identification of overall gene expression profiles or select up- or down-regulated genes associated with any particular combination of antigen specificities.

In some embodiments, the method of any preceding aspect further comprises identification of surface markers, via, for example, fluorescence-activated cell sorting, or oligo-conjugated antibodies associated with any particular combination of antigen specificities

In some embodiments, the method of any preceding aspect further comprises identification of any combination of BCR sequence feature (for example, immunoglobulin gene, sequence motif, or CDR length), gene expression profile, or surface marker profile associated with any particular combination of antigen specificities.

In some embodiments, the method of any preceding aspect further comprises training a machine learning algorithm on sequence features, sequence motifs, or encoded sequence properties (such as via Kidera factors), associated with any particular combination of antigen specificities for subsequent application to sequenced antibodies lacking antigen specificity information due to not using LIBRA-seq or otherwise.

In some aspects, disclosed herein is a method for simultaneous detection of an antigen and an antibody that specifically binds said antigen, comprising:
labeling a plurality of antigens with unique antigen barcodes;
providing a plurality of barcode-labeled antigens to a population of B-cells;
allowing the plurality of barcode-labeled antigens to bind to the population of B-cells;
washing unbound antigens from the population of B-cells;
separating the B-cells into single cell emulsions;
introducing into each single cell emulsion a unique cell barcode-labeled bead;
preparing a single cell cDNA library from the single cell emulsions;
performing PCR amplification reactions to produce a plurality of amplicons, wherein the amplicons comprise: 1) the cell barcode and the antigen barcode, 2) the cell barcode and an antibody sequence, and 3) a unique molecular identifier (UMI);
sequencing the plurality of amplicons;
removing a sequence lacking the cell barcode, the UMI, or the antigen barcode;
aligning the antibody sequence to a reference library of immunoglobulin V, D, J and C sequences;
constructing a UMI count matrix comprising the cell barcode, the antigen barcode, and the antibody sequence;
determining a LIBRA-seq score; and
determining that the antibody specifically binds an antigen if the LIBRA-seq score of the antibody for the antigen is increased in comparison to a control sample.

In some embodiments, the barcode-labeled antigens are labeled with a first barcode comprising a DNA sequence or an RNA sequence. In some embodiments, the cell barcode-labeled beads are labeled with a second barcode comprising a DNA sequence or an RNA sequence.

It should be understood that the barcode described above is conjugated to the barcode-labeled antigen in a way that are known to one of ordinary skill in the art. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. An oligonucleotide barcode can also be conjugated to an antigen using the Solulink Protein-Oligonucleotide Conjugation Kit (TriLink cat no. S-9011) according to manufacturer's instructions. Briefly, the oligo and protein are desalted, and then the amino-oligo is modified with the 4FB crosslinker, and the biotinylated antigen protein is modified with S-HyNic. Then, the 4FB-oligo and the HyNic-antigen are mixed together. This causes a stable bond to form between the protein and the oligonucleotide. In some embodiments, the cell barcode-labeled beads are labeled with a second barcode comprising a DNA sequence or an RNA sequence. In some embodiments, the cell barcode-labeled beads are labeled with a second barcode comprising a DNA sequence. In some embodiments, the cell barcode-labeled beads are labeled with a second barcode comprising an RNA sequence. In some embodiments, the cell barcode-labeled beads are labeled with a barcode on the inside of the bead. In some embodiments, the cell barcode-labeled beads are labeled with a barcode encapsulated within the bead. In some embodiments, the cell barcode-labeled beads are labeled with a barcode on the outside of the bead.

As used herein, “beads” is not limited to a specific type of bead. Rather, a large number of beads are available and are known to one of ordinary skill in the art. A suitable bead may be selected on the basis of the desired end use and suitability for various protocols. In some embodiments, the bead is or comprises a particle or a bead. In some embodiments, the solid support bead is magnetic. Beads comprise particles have been described in the prior art in, for example, U.S. Pat. Nos. 5,084,169, 5,079,155, 473,231, and 8,110,351. The particle or bead size can be optimized for binding B cell in a single cell emulsion and optimized for the subsequent PCR reaction.

These oligos, which contain the cell barcode, both: (1) enable amplification of cellular mRNA transcripts through the template switch oligo that is part of the oligo containing the cell barcode, and (2) directly anneal to the antigen barcode-containing oligos from the antigen. In some embodiments, the oligos delivered from the beads have the general structure: P5_PCR_handle-Cell_barcode-UMI-Template_switch_oligo.

It is noted above that the antibody is determined as specifically binding an antigen if the LIBRA-seq score of the antibody for the antigen is increased in comparison to a control sample. It should be understood herein that, as taught by FIG. 1C, between the minimum (y-axis, top) and maximum (y-axis, bottom) LIBRA-seq score for each antigen, the ability of each of 100 cutoffs was tested for its ability to classify each antibody as antigen positive or negative, where antigen positive is defined as having a LIBRA-seq score greater than or equal to the cutoff being evaluated and antigen negative is defined as having a LIBRA-seq score below the cutoff.

In some embodiments, the antibody sequence comprises an immunoglobulin heavy chain (VDJ) sequence, or an immunoglobulin light chain (VJ) sequence. In some embodiments, the antibody sequence comprises an immunoglobulin heavy chain (VDJ) sequence. In some embodiments, the antibody sequence comprises an immunoglobulin light chain (VJ) sequence.

In some embodiments, the barcode-labeled antigens comprise an antigen from a pathogen or an animal In some embodiments, the barcode-labeled antigens comprise an antigen from a pathogen. In some embodiments, the barcode-labeled antigens comprise an antigen from an animal In some embodiments, the animal is a mammal, including, but not limited to, primates (e.g., humans and nonhuman primates), cows, sheep, goats, horses, dogs, cats, rabbits, rats, mice and the like. In some embodiments, the subject is a human.

In some embodiments, the antigen from a pathogen comprises an antigen from a virus. In some embodiments, the antigen from a virus comprises an antigen from human immunodeficiency virus (HIV), an antigen from influenza virus, or an antigen from respiratory syncytial virus (RSV).

In some embodiments, the antigen from a virus comprises an antigen from human immunodeficiency virus (HIV). In some embodiments, the antigen from a virus comprises an antigen from influenza virus. In some embodiments, the antigen from a virus comprises an antigen from respiratory syncytial virus (RSV).

In some embodiments, the antigen from HIV comprises an antigen from HIV-1. In some embodiments, the antigen from HIV comprises an antigen from HIV-2. In some embodiments, the antigen from HIV comprises HIV-1 Env. In some embodiments, the antigen from influenza virus comprises hemagglutinin (HA). In some embodiments, the antigen from RSV comprises an RSV F protein. In some embodiments, the antigen is selected from the antigens listed in Table 1.

TABLE 1
Antigen screening library for human B-cell sample analysis. For a set of pathogens,
shown are selected protein targets, number of strains, and resulting total number of
antigens in the screening library.

| Pathogen | Protein targets | # Strains | # Antigens in library |
|---|---|---|---|
| CMV | g^{B} | 2 | 2 |
| D [symbol] ngue | E, prM | 6 | 10 |
| Hepatitis B | HBsAg | 2 | 2 |
| Hepatitis C | E2, E1E2 | 2 | 4 |
| HIV-1 | gp140, gp120, MPER | 3 | 9 |
| HPV | L1 | 3 | 3 |
| HSV-1 | g^{B} | 1 | 1 |
| influenza | HA [symbol] NA | [symbol] | 12 |
| Malaria | PfCSP | 1 | 1 |
| Measles | H, F | 1 | 2 |
| Mumps | HN, NP | 1 | 2 |
| Norovirus | P | 10 | 10 |
| Rhinovius | VP1 | 5 | 5 |
| Rotavirus | VP7, VP4 | [symbol] | 8 |
| RSV | F [symbol] G | 4 | 8 |
| Rub [symbol] a | E1 | 1 | 1 |
| Staphylococcus aureus | HtsA, SirA, IsdB, SstD | 1 | 4 |
| UPEC | Hma, IutA, FyuA, IreA | 1 | 4 |
| Z [symbol] ka | E [symbol] prM | 1 | 2 |

*influenza: A (6 HA, 4 NA) and B (2 HA); \hat{ }rotavirus: 6 G, 2 P variants) [symbol]
indicates data missing or illegible when filed

In some embodiments, the population of B-cells comprise a memory B-cell, a plasma cell, a naïve B cell, an activated B-cell, or a B-cell line. In some embodiments, the population of B-cells comprise a memory B-cell, a plasma cell, a naïve B cell, an activated B-cell, or a B-cell line. In some embodiments, the population of B-cells comprise a plasma cell. In some embodiments, the population of B-cells comprise a naïve B cell. In some embodiments, the population of B-cells comprise an activated B-cell. In some embodiments, the population of B-cells comprise a B-cell line.

In another aspect, disclosed herein is a method of determining a broadly neutralizing antibody to a pathogen, said method comprising:
labeling a plurality of antigens derived from the pathogen with unique antigen barcodes;
providing a plurality of barcode-labeled antigens to a population of B-cells;
allowing the plurality of barcode-labeled antigens to bind to the population of B-cells;
washing unbound antigens from the population of B-cells;
separating the B-cells into single cell emulsions;
introducing into each single cell emulsion a unique cell barcode-labeled bead;
preparing a single cell cDNA library from the single cell emulsions;
performing PCR amplification reactions to produce a plurality of amplicons, wherein the amplicons comprise: 1) the cell barcode and the antigen barcode, 2) the cell barcode and an antibody sequence, and 3) a unique molecular identifier (UMI);
sequencing the plurality of amplicons;
removing a sequence lacking a cell barcode, unique molecular identifier (UMI), or an antigen barcode;
aligning the antibody sequence to a reference library of immunoglobulin V, D, J and C sequences;
constructing a UMI count matrix comprising the cell barcode, the antigen barcode, and the antibody sequence;
determining a LIBRA-seq score; and
determining that the antibody is a broadly neutralizing antibody if the LIBRA-seq scores of the antibody for two or more antigens are increased in comparison to a control.

Polypeptides and Polynucleotides

In some aspects, disclosed herein is a polynucleotide comprising a sequence set forth in the specification.

In some aspects, disclosed herein is a polypeptide, wherein the polypeptide is encoded by a polynucleotide sequence set forth in the specification.

In some aspects, disclosed herein is a recombinant antibody, said antibody comprising a light chain variable region (VL) and a heavy chain variable region (VH), wherein
the VH comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 667-711; and/or
the VL comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 802-845.

In some embodiments, the VH comprises at least one amino acid substitution (including, for example, at least 1, 2, 3, 4, 5, 6, 7, 8, 9, 10, 11, 12, 13, 14, 15, or 16 substitutions) when compared to SEQ ID NOs: 667-711. In some embodiments, the VL comprises at least one amino acid substitution (including, for example, at least 1, 2, 3, 4, 5, 6, 7, 8, 9, 10, 11, 12, 13, 14, 15, or 16 substitutions) when compared to SEQ ID NOs: 802-845.

In some aspects, disclosed herein is a recombinant antibody, said antibody comprising a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein
the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 712-740; and/or
the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 846-876.

In some aspects, disclosed herein is a recombinant antibody, said antibody comprising a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein
the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 741-767; and/or
the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 877-891.

In some aspects, disclosed herein is a recombinant antibody, said antibody comprising a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein
the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 768-801 or 917-936; and/or
the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 892-916 or 937-938.

In some aspects, disclosed herein is a recombinant antibody, said antibody comprising a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein
the CDRH1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 712-740;
the CDRL1 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 846-876;
the CDRH2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 741-767;
the CDRL2 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 877-891;
the CDRH3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 768-801 or 917-936; and/or
the CDRL3 comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 892-916 or 937-938.

In some embodiments, the CDRH1 comprises at least one amino acid substitution (including, for example, at least 1, 2, 3, 4, 5, or 6 substitutions) when compared to SEQ ID NOs: 712-740. In some embodiments, the CDRH2 comprises at least one amino acid substitution (including, for example, at least 1, 2, 3, 4, 5, or 6 substitutions) when compared to SEQ ID NOs: 741-767. In some embodiments, the CDRH3 comprises at least one amino acid substitution (including, for example, at least 1, 2, 3, 4, 5, or 6 substitutions) when compared to SEQ ID Nos: 768-801 or 917-936. In some embodiments, the CDRH3 comprises at least one amino acid substitution (including, for example, at least 1, 2, 3, 4, 5, or 6 substitutions) when compared to SEQ ID NO: 770. In some embodiments, the CDRH3 comprises at least one amino acid substitution (including, for example, at least 1, 2, 3, 4, 5, or 6 substitutions) when compared to SEQ ID NO: 771. In some embodiments, the CDRH3 comprises at least one amino acid substitution (including, for example, at least 1, 2, 3, 4, 5, or 6 substitutions) when compared to SEQ ID NO: 917. In some embodiments, the CDRH3 comprises at least one amino acid substitution (including, for example, at least 1, 2, 3, 4, 5, or 6 substitutions) when compared to SEQ ID NO: 918. In some embodiments, the CDRH3 comprises at least one amino acid substitution (including, for example, at least 1, 2, 3, 4, 5, or 6 substitutions) when compared to SEQ ID NO: 919. In some embodiments, the CDRH3 comprises at least one amino acid substitution (including, for example, at least 1, 2, 3, 4, 5, or 6 substitutions) when compared to SEQ ID NO: 920. In some embodiments, the CDRH3 comprises at least one amino acid substitution (including, for example, at least 1, 2, 3, 4, 5, or 6 substitutions) when compared to SEQ ID NO: 921. In some embodiments, the CDRH3 comprises at least one amino acid substitution (including, for example, at least 1, 2, 3, 4, 5, or 6 substitutions) when compared to SEQ ID NO: 922. In some embodiments, the CDRH3 comprises at least one amino acid substitution (including, for example, at least 1, 2, 3, 4, 5, or 6 substitutions) when compared to SEQ ID NO: 923. In some embodiments, the CDRH3 comprises at least one amino acid substitution (including, for example, at least 1, 2, 3, 4, 5, or 6 substitutions) when compared to SEQ ID NO: 924. In some embodiments, the CDRH3 comprises at least one amino acid substitution (including, for example, at least 1, 2, 3, 4, 5, or 6 substitutions) when compared to SEQ ID NO: 925. In some embodiments, the CDRH3 comprises at least one amino acid substitution (including, for example, at least 1, 2, 3, 4, 5, or 6 substitutions) when compared to SEQ ID NO: 926. In some embodiments, the CDRH3 comprises at least one amino acid substitution (including, for example, at least 1, 2, 3, 4, 5, or 6 substitutions) when compared to SEQ ID NO: 927. In some embodiments, the CDRH3 comprises at least one amino acid substitution (including, for example, at least 1, 2, 3, 4, 5, or 6 substitutions) when compared to SEQ ID NO: 928. In some embodiments, the CDRH3 comprises at least one amino acid substitution (including, for example, at least 1, 2, 3, 4, 5, or 6 substitutions) when compared to SEQ ID NO: 929. In some embodiments, the CDRH3 comprises at least one amino acid substitution (including, for example, at least 1, 2, 3, 4, 5, or 6 substitutions) when compared to SEQ ID NO: 930. In some embodiments, the CDRH3 comprises at least one amino acid substitution (including, for example, at least 1, 2, 3, 4, 5, or 6 substitutions) when compared to SEQ ID NO: 931. In some embodiments, the CDRH3 comprises at least one amino acid substitution (including, for example, at least 1, 2, 3, 4, 5, or 6 substitutions) when compared to SEQ ID NO: 932. In some embodiments, the CDRH3 comprises at least one amino acid substitution (including, for example, at least 1, 2, 3, 4, 5, or 6 substitutions) when compared to SEQ ID NO: 933. In some embodiments, the CDRH3 comprises at least one amino acid substitution (including, for example, at least 1, 2, 3, 4, 5, or 6 substitutions) when compared to SEQ ID NO: 934. In some embodiments, the CDRH3 comprises at least one amino acid substitution (including, for example, at least 1, 2, 3, 4, 5, or 6 substitutions) when compared to SEQ ID NO: 935. In some embodiments, the CDRH3 comprises at least one amino acid substitution (including, for example, at least 1, 2, 3, 4, 5, or 6 substitutions) when compared to SEQ ID NO: 936. In some embodiments, the CDRH3 comprises a polypeptide sequence selected from SEQ ID NOs: 770-771 or 917-936.

In some embodiments, the CDRL1 comprises at least one amino acid substitution (including, for example, at least 1, 2, 3, 4, 5, or 6 substitutions) when compared to SEQ ID NOs: 846-876. In some embodiments, the CDRL2 comprises at least one amino acid substitution (including, for example, at least 1, 2, 3, 4, 5, or 6 substitutions) when compared to SEQ ID NOs: 877-891. In some embodiments, the CDRL3 comprises at least one amino acid substitution (including, for example, at least 1, 2, 3, 4, 5, or 6 substitutions) when compared to SEQ ID NOs: 892-916 or 937-938. In some embodiments, the CDRL3 comprises at least one amino acid substitution (including, for example, at least 1, 2, 3, 4, 5, or 6 substitutions) when compared to SEQ ID NO: 894. In some embodiments, the CDRL3 comprises at least one amino acid substitution (including, for example, at least 1, 2, 3, 4, 5, or 6 substitutions) when compared to SEQ ID NO: 895. In some embodiments, the CDRL3 comprises at least one amino acid substitution (including, for example, at least 1, 2, 3, 4, 5, or 6 substitutions) when compared to SEQ ID NO: 896. In some embodiments, the CDRL3 comprises at least one amino acid substitution (including, for example, at least 1, 2, 3, 4, 5, or 6 substitutions) when compared to SEQ ID NO: 897. In some embodiments, the CDRL3 comprises at least one amino acid substitution (including, for example, at least 1, 2, 3, 4, 5, or 6 substitutions) when compared to SEQ ID NO: 902. In some embodiments, the CDRL3 comprises at least one amino acid substitution (including, for example, at least 1, 2, 3, 4, 5, or 6 substitutions) when compared to SEQ ID NO: 903. In some embodiments, the CDRL3 comprises at least one amino acid substitution (including, for example, at least 1, 2, 3, 4, 5, or 6 substitutions) when compared to SEQ ID NO: 904. In some embodiments, the CDRL3 comprises at least one amino acid substitution (including, for example, at least 1, 2, 3, 4, 5, or 6 substitutions) when compared to SEQ ID NO: 905. In some embodiments, the CDRL3 comprises at least one amino acid substitution (including, for example, at least 1, 2, 3, 4, 5, or 6 substitutions) when compared to SEQ ID NO: 906. In some embodiments, the CDRL3 comprises at least one amino acid substitution (including, for example, at least 1, 2, 3, 4, 5, or 6 substitutions) when compared to SEQ ID NO: 907. In some embodiments, the CDRL3 comprises at least one amino acid substitution (including, for example, at least 1, 2, 3, 4, 5, or 6 substitutions) when compared to SEQ ID NO: 908. In some embodiments, the CDRL3 comprises at least one amino acid substitution (including, for example, at least 1, 2, 3, 4, 5, or 6 substitutions) when compared to SEQ ID NO: 911. In some embodiments, the CDRL3 comprises at least one amino acid substitution (including, for example, at least 1, 2, 3, 4, 5, or 6 substitutions) when compared to SEQ ID NO: 915. In some embodiments, the CDRL3 comprises at least one amino acid substitution (including, for example, at least 1, 2, 3, 4, 5, or 6 substitutions) when compared to SEQ ID NO: 937. In some embodiments, the CDRL3 comprises at least one amino acid substitution (including, for example, at least 1, 2, 3, 4, 5, or 6 substitutions) when compared to SEQ ID NO: 938. In some embodiments, the CDRL3 comprises a polypeptide sequence selected from the group consisting of SEQ ID NOs: 894-897, 902-908, 911, 915, 937, or 938.

In some aspects, disclosed herein is a recombinant antibody, said antibody comprising a heavy chain variable region (VH) that comprises a VDJ junction, wherein
the VDJ junction comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 775 or 939-948.

In some aspects, disclosed herein is a recombinant antibody, said antibody comprising a light chain variable region (VL) that comprises a VJ junction, wherein
the VJ junction comprises an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 892, 893, 899, 900, 909, 910, 912, 913, 914, or 916.

In some aspects, disclosed herein is a recombinant antibody, said antibody comprising a heavy chain variable region (VH) and a light chain variable region (VL), wherein the VH comprises a VDJ junction comprising an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 775 or 939-948, and wherein the VL comprises a VJ junction comprising an amino acid sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 892, 893, 899, 900, 909, 910, 912, 913, 914, or 916.

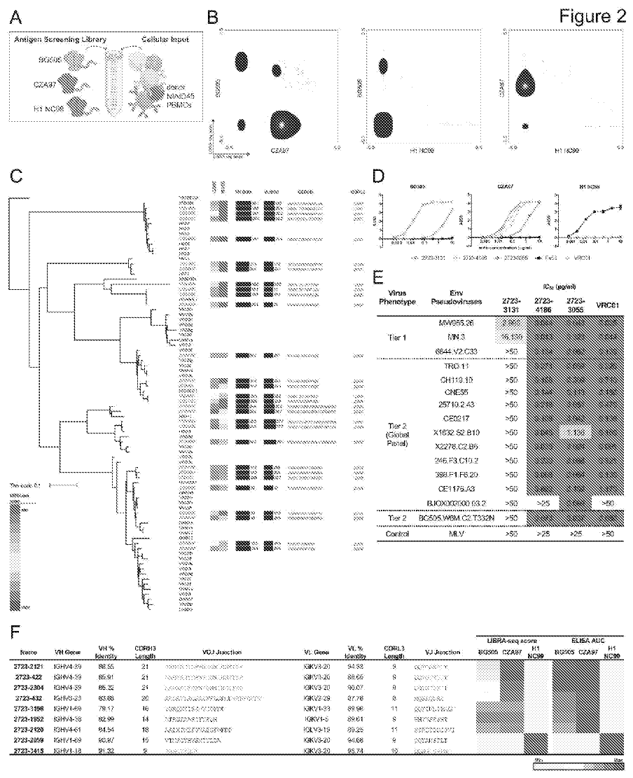

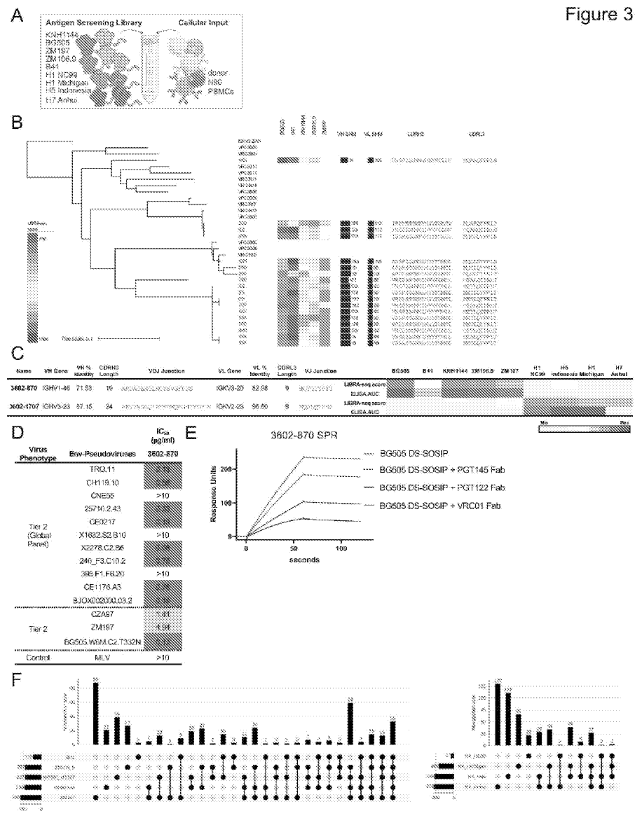

In some aspects, disclosed herein is a polypeptide comprising a sequence set forth in FIG. 2 or FIG. 3. In some aspects, disclosed herein is a recombinant antibody comprising a sequence set forth in FIG. 2 or FIG. 3.

In some aspects, disclosed herein is a recombinant antibody, said antibody comprising a heavy chain variable region (VH) that is encoded by a polynucleotide at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 223-444.

In some aspects, disclosed herein is a recombinant antibody, said antibody comprising a light chain variable region (VL) that is encoded by a polynucleotide at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 445-666.

In some aspects, disclosed herein is a recombinant antibody, said antibody comprising a heavy chain variable region (VH) and a light chain variable region (VL), wherein the VH is encoded by a polynucleotide at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 223-444, and wherein the VL is encoded by a polynucleotide at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NOs: 445-666.

In some aspects, disclosed herein is a therapeutic antibody comprising the polypeptide of any preceding aspect. The term “neutralizing antibody” is any antibody or antigen-binding fragment thereof that binds to a pathogen and interferes with the ability of the pathogen to infect a cell and/or cause disease in a subject. Typically, the neutralizing antibodies used in the method of the present disclosure bind to the surface of the pathogen and inhibit or reduce infection by the pathogen by at least 99 percent, at least 95 percent, at least 90 percent, at least 85 percent, at least 80 percent, at least 75 percent, at least 70 percent, at least 60 percent, at least 50 percent, at least 45 percent, at least 40 percent, at least 35 percent, at least 30 percent, at least 25 percent, at least 20 percent, or at least 10 percent relative to infection by the pathogen (e.g., HIV or influenza) in the absence of said antibody(ies) or in the presence of a negative control.

In some embodiments, the neutralizing antibody comprises a polypeptide sequence set forth in the specification. In some embodiments, the neutralizing antibody comprises 3602-870, or a polypeptide sequence having at or greater than about 80%, about 85%, about 90%, about 95%, or about 98% homology with the sequence of 3602-870, or a polypeptide comprising a portion of 3602-870. As used herein, “broadly neutralizing antibody” or “BNAb” is understood as an antibody obtained by any method that when delivered at an effective dose can be used as a therapeutic agent for the prevention or treatment of HIV or influenza infection or an infection-related disease against a broad array of different HIV or influenza strains (for example, more than 3 strains of HIV/influenza, preferably more than 4, 5, 6, 7, 8, 9, 10, 11, 12, 13, 14, 15, 16, 17, 18, 19, 20, or more strains of HIV/influenza). In some embodiments, the broadly neutralizing antibody comprises a polypeptide sequence set forth in the specification. In some embodiments, the neutralizing antibody comprises 3602-870, or a polypeptide sequence having at or greater than about 80%, about 85%, about 90%, about 95%, or about 98% homology with the sequence of 3602-870, or a polypeptide comprising a portion of 3602-870.

Accordingly, in some embodiments, the neutralizing antibody comprises a VH and a VL, wherein the VH comprises a polypeptide sequence at least 60% (for example, at least 60%, at least 65%, at least 70%, at least 75%, at least 80%, at least 85%, at least 90%, at least 95%, at least 96%, at least 97%, at least 98%, at least 99%) to SEQ ID NO: 685, and wherein the VL comprises a polypeptide sequence at least 60% (for example, at least 60%, at least 65%, at least 70%, at least 75%, at least 80%, at least 85%, at least 90%, at least 95%, at least 96%, at least 97%, at least 98%, at least 99%) to SEQ ID NO: 813. In some embodiments, the neutralizing antibody comprises a VH comprising a CDRH1, CDRH2, and CDRH3, wherein the CDRH1 comprises a polypeptide sequence at least 60% (for example, at least 60%, at least 65%, at least 70%, at least 75%, at least 80%, at least 85%, at least 90%, at least 95%, at least 96%, at least 97%, at least 98%, at least 99%) to SEQ ID NO: 713, wherein the CDRH2 comprises a polypeptide sequence at least 60% (for example, at least 60%, at least 65%, at least 70%, at least 75%, at least 80%, at least 85%, at least 90%, at least 95%, at least 96%, at least 97%, at least 98%, at least 99%) to SEQ ID NO: 749, and wherein the CDRH3 comprises a polypeptide sequence at least 60% (for example, at least 60%, at least 65%, at least 70%, at least 75%, at least 80%, at least 85%, at least 90%, at least 95%, at least 96%, at least 97%, at least 98%, at least 99%) to SEQ ID NO: 773. In some embodiments, the neutralizing antibody comprises a VL comprising a CDRL1, CDRL2, and CDRL3, wherein the CDRL1 comprises a polypeptide sequence at least 60% (for example, at least 60%, at least 65%, at least 70%, at least 75%, at least 80%, at least 85%, at least 90%, at least 95%, at least 96%, at least 97%, at least 98%, at least 99%) to SEQ ID NO: 851, wherein the CDRL2 comprises a polypeptide sequence at least 60% (for example, at least 60%, at least 65%, at least 70%, at least 75%, at least 80%, at least 85%, at least 90%, at least 95%, at least 96%, at least 97%, at least 98%, at least 99%) to SEQ ID NO: 879, and wherein the CDRL3 comprises a polypeptide sequence at least 60% (for example, at least 60%, at least 65%, at least 70%, at least 75%, at least 80%, at least 85%, at least 90%, at least 95%, at least 96%, at least 97%, at least 98%, at least 99%) to SEQ ID NO: 893.

In some aspect, disclosed herein is a method of treating HIV infection in a subject, comprising administering to the subject a therapeutically effective amount of the recombinant polypeptide and/or neutralizing antibody of any preceding aspect.

In some aspect, disclosed herein is a method of treating flu infection in a subject, comprising administering to the subject a therapeutically effective amount of the recombinant polypeptide and/or neutralizing antibody of any preceding aspect.

### EXAMPLES

The following examples are set forth below to illustrate the systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. LIBRA-seq Method

LIBRA-seq transforms antibody-antigen interactions into sequencing-detectable events by conjugating DNA-barcoded oligos to each antigen in a screening library. All antigens are labeled with the same fluorophore, which enables sorting of antigen-positive B cells by fluorescence-activated cell sorting (FACS) before encapsulation of single B cells via droplet microfluidics. Antigen barcodes and BCR transcripts are tagged with a common cell barcode from bead-delivered oligos, enabling direct mapping of BCR sequence to antigen specificity (FIG. 1A).

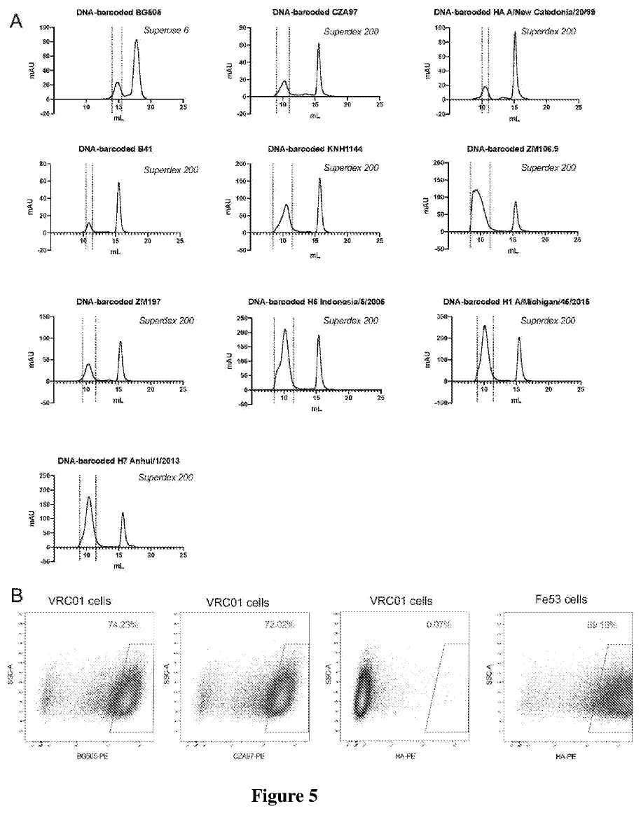

To investigate the ability of LIBRA-seq to accurately unite BCR sequence and antigen specificity, a mapping experiment was devised using two Ramos B-cell lines with differing BCR sequences and antigen specificities (Weaver et al., 2016). These engineered B-cell lines do not display endogenous BCR and instead express specific, user-defined surface IgM BCR sequences (Weaver et al., 2016). To that end, two well-characterized BCRs were selected: VRC01, a CD4-binding site-directed HIV-1 bNAb (Wu et al., 2010), and Fe53, a bNAb recognizing the stem of group 1 influenza hemagglutinins (HA) (Lingwood et al., 2012). These two populations of B -cell lines were mixed at a 1:1 ratio and incubated with three unique DNA-barcoded antigens: two variants of the trimeric HIV-1 Env protein from strains BG505 and CZA97 (Georgiev et al., 2015; van Gils et al., 2013; Ringe et al., 2017), and trimeric hemagglutinin from strain H1 A/New Caledonia/20/1999 (Whittle et al., 2014) (FIG. 1B; FIGS. 5A-B and 6A).

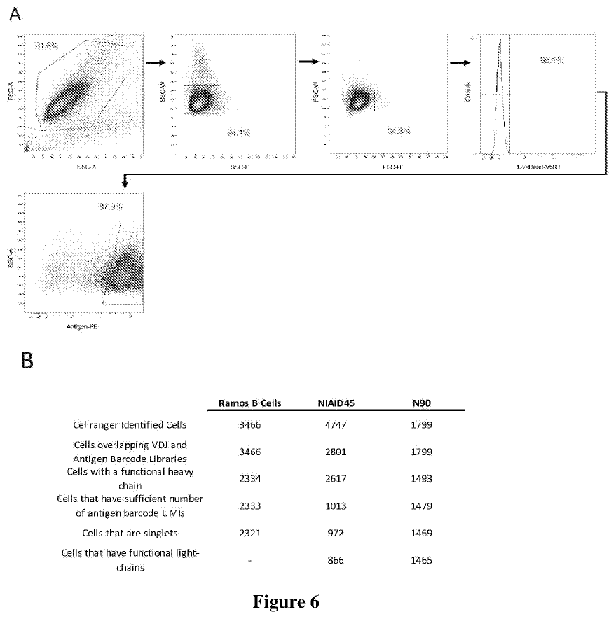

2321 cells with BCR sequence and antigen mapping information were recovered, highlighting the high throughput capacity of LIBRA-seq (FIG. 6B). For each cell, the LIBRA-seq scores for each antigen in the screening library were computed as a function of the number of unique molecular identifiers (UMIs) for the respective antigen barcode; therefore, scores serve as a proxy for the relative amount of bound antigen (Methods). The LIBRA-seq scores of each individual antigen reliably categorized Ramos B cells by their specificity (FIG. 1C). Overall, cells fell into two major populations based on their LIBRA-seq scores, and no cell was observed with cross-reactivity for influenza HA and HIV-1 Env (FIG. 1D). Further, VRC01 Ramos B cells bound both BG505 and CZA97 with a high correlation between the scores for these two antigens (Pearson's r=0.84), showing that LIBRA-seq readily identifies B cells that bind to multiple HIV-1 antigens (FIG. 1E).

### Example 2. Isolation of Antibodies from a Known HIV bNAb Lineage

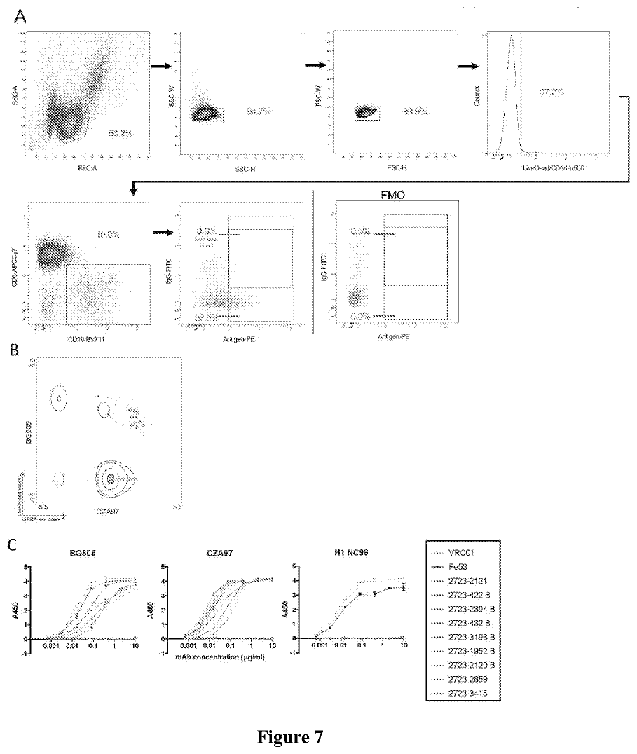

LIBRA-seq was next used to analyze the antibody repertoire of donor NIAID 45, who had been living with HIV-1 without antiretroviral therapy for approximately 17 years at the time of sample collection. This sample was selected as an appropriate target for LIBRA-seq analysis because a large lineage of HIV-1 bNAbs had been identified previously from this donor (Bonsignori et al., 2018; Wu et al., 2010, 2015). This lineage consists of the prototypical bNAb VRC01, as well as multiple clades of clonally related bNAbs with diverse neutralization phenotypes (Wu et al., 2015). The same BG505, CZA97, and H1 A/New Caledonia/20/99 antigen screening library was used in the Ramos B-cell line experiments, recovering paired V_{H}:V_{L} antibody sequences with antigen mapping for 866 cells (FIG. 2A; FIGS. 6B and 7A). These B cells exhibited a variety of LIBRA-seq scores among the three antigens (FIG. 2B), as these were from a polyclonal sample possessing a wide variety of B cell specificities and antigen affinities. The cells displayed a few discrete patterns based on their LIBRA-seq scores; generally, cells were either (1) HA^{high}Env^{low} or (2) HA^{low}Env^{high} (FIG. 2B). Additionally, cells that were double positive for both HIV Env variants, BG505 and CZA97 were observed, indicating HIV-1 strain cross-reactivity of these B cells (FIG. 2B).

To further investigate LIBRA-seq in monoclonal antibody isolation, new members of the VRC01 antibody lineage were identified from the LIBRA-seq-identified antigen-specific B cells. 29 BCRs that were clonally related to previously-identified members of the VRC01 lineage (FIG. 2C) were observed. All newly identified BCRs had high levels of somatic hypermutation and utilized IGHV1-2*02 along with the characteristic five-residue CDRL3 paired with IGVK3-20 (FIG. 2D). These B cells came from multiple known clades of the VRC01 lineage, with sequences with high identity and phylogenetic relatedness to lineage members VRC01, VRC02, VRC03, VRC07, VRC08, NIH45-46, and others (FIG. 2C). Of these, 25 (87%) had a high LIBRA-seq score for at least 1 HIV-1 antigen, three (10%) had mid-range scores (between 0 and 1) for at least 1 HIV-1 antigen, and only one of the VRC01 lineage B cells had negative scores for both HIV-1 antigens (FIG. 2C, FIG. 7B). Three of the newly identified lineage members, named 2723-3055, 2723-4186 and 2723-3131, were recombinantly expressed to confirm the ability of these antibodies to bind the screening probes. 2723-3131 bound to CZA97 and had somewhat lower binding to BG505 by enzyme linked immunosorbent assay (ELISA) (FIG. 2D). 2723-3131 did not neutralize any viruses on the global panel (deCamp et al., 2014) but did neutralize two Tier one viruses (FIG. 2E). Both 2723-3055 and 2723-4186 bound to BG505 and CZA97, and potently neutralized 11/12 and 12/12 viruses on a global panel, respectively (FIG. 2D-2E). Together, the results from the donor 45 analysis show that the LIBRA-seq platform can be successfully used to down-select cross-reactive bNAbs in prospective antibody discovery efforts.

### Example 3. Identification of Additional Broadly-Reactive Anti-HIV and Anti-Influenza Antibodies

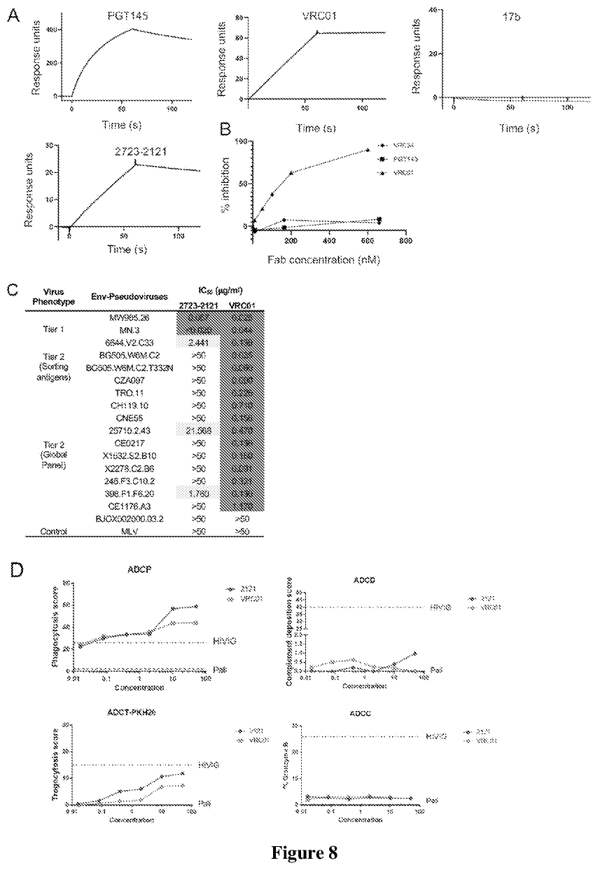

To further assess the ability of LIBRA-seq to accurately identify antigen-specific B cells, a number of putative HIV-specific and influenza-specific monoclonal antibodies were produced from donor 45 that did not belong to the VRC01 lineage. In particular, seven additional anti-HIV antibodies were recombinantly produced, three of which were clonally related (2723-2121, 2723-422, and 2723-2304) (FIG. 2F). These seven antibodies were selected because all had high LIBRA-seq scores for at least one HIV-1 antigen. All seven antibodies bound the antigens by ELISA based on the respective LIBRA-seq scores, with high similarity between the patterns of LIBRA-seq scores and ELISA area under the curve (AUC) values (FIG. 2F, FIG. 7C, Methods). One of these antibodies, 2723-2121, were characterized, determining that it bound to a stabilized BG505 trimer (Do Kwon et al., 2015) by surface plasmon resonance (SPR) (FIG. 8A), was indicated to have a CD4 binding site epitope specificity (FIG. 8B), neutralized three Tier 1 pseudoviruses and 2/11 Tier 2 pseudoviruses from the global panel (FIG. 8C), and mediated trogocytosis and antibody-dependent cellular phagocytosis (FIG. 8D). In addition to the HIV-specific antibodies, assessment was performed to characterize two antibodies predicted of having influenza specificity based on their LIBRA-seq scores for H1 A/New Caledonia/20/99 (FIG. 2F). In agreement with the LIBRA-seq scores, antibodies 2723-2859 and 2723-3415 bound H1 A/New Caledonia/20/99 but not BG505 or CZA97 by ELISA, confirming the ability of LIBRA-seq to simultaneously isolate antibodies to multiple diverse antigens (FIG. 2F, FIG. 7C).

### Example 4. Discovery of an HIV bNAb using a Nine-Antigen Screening Library

Having validated LIBRA-seq with three antigens on both Ramos B cell lines and primary B cells from a patient sample, experiment was performed to increase the number of antigens in the screening library. To that end, the B cell repertoire of NIAID donor N90 was screened against nine antigens (FIG. 3A). This sample was selected because a single broadly neutralizing antibody lineage (VRC38) targeting the V1/V2 epitope was isolated previously from this donor; however, the neutralization breadth of the VRC38 lineage could not account for the full serum neutralization breadth (Cale et al., 2017; Wu et al., 2012). This suggests that there could be additional bNAb lineages present in the B cell repertoire of N90 and that utilizing multiple SOSIP probes could help accelerate identification of such antibodies. Thus, whether LIBRA-seq can accomplish two goals was determined: (1) to recover antigen-specific B cells from the VRC38 lineage, and (2) to identify new bNAbs that can neutralize viruses that are resistant to the VRC38 lineage but sensitive to the serum.

To increase the number of antigens in the screening library, a panel consisted of five HIV-1 Env trimers from a variety of clades, BG505 (clade A), B41 (clade B), ZM106.9 (clade C), ZM197 (clade C) and KNH1144 (clade A) was utilized (van Gils et al., 2013; Harris et al., 2011; Joyce et al., 2017; Julien et al., 2015; Pugach et al., 2015; Ringe et al., 2017), along with four diverse hemagglutinin trimers (H1 A/New Caledonia/20/99, H1 A/Michigan/45/2015, H5 A/Indonesia/5/2005, and H7 A/Anhui/1/2013) (FIG. 3A, FIG. 5A). After applying LIBRA-seq to donor N90 PBMCs, paired V_{H}:V_{L} antibody sequences with antigen mapping for 1465 cells (FIG. 6B, 9A) were recovered. Within this set of cells, eighteen B cells were identified as members of the VRC38 lineage (FIG. 3B). Of these, seventeen had high LIBRA-seq scores for at least one HIV antigen, and one had no high LIBRA-seq scores but had a mid-range score for two SOSIPs (FIG. 3B), indicating that LIBRA-seq can successfully identify HIV-1 reactivity for virtually all B cells from the VRC38 lineage.

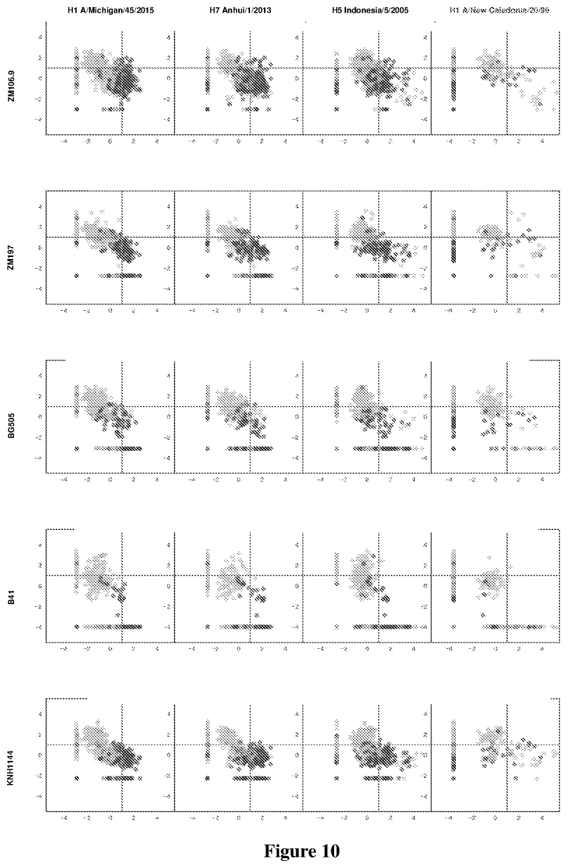

The B cells with the highest LIBRA-seq scores in the N90 sample were analyzed, especially those cells that had LIBRA-seq scores for any antigen above one (901 cells) (FIG. 10). 32 cells were observed with high LIBRA-seq scores for three of the four influenza antigens (FIG. 3F); one of these, 3602-1707, was recombinantly produced and confirmed with broad influenza recognition, with high correlation between LIBRA-seq scores and ELISA AUC (Spearman correlation 0.77, p=0.015) (FIG. 3C, FIG. 9B).

Cells that had high LIBRA-seq scores for each of multiple HIV-1 antigens were also observed, including 124 cells that had high scores for four or more SOSIPs (FIG. 3F). SOSIP-high B cells were then down selected based on two requirements: (1) high LIBRA-seq scores to at least 3 SOSIP variants, and (2) one of these SOSIP variants must be ZM106.9, since the serum of N90 neutralized ZM106.9 but the VRC38 lineage did not (Cale et al., 2017). In particular, two members from the same antibody lineage were identified with high LIBRA-seq scores for BG505, KNH1144, ZM106.9 and ZM197. This lineage utilized the germline genes IGHV1-46 and IGK3-20, was highly mutated in both the heavy- and light-chain V genes, and had a 19 amino acid CDRH3 and nine amino acid CDRL3. One of the lineage members, 3602-870, that was 28.5% mutated in its heavy chain V gene and 17.0% mutated in its light chain V gene (FIG. 3C) was recombinantly expressed. 3602-870 bound all SOSIP probes by ELISA (Spearman correlation of 0.97, p<0.001 between LIBRA-seq scores and ELISA AUC) and neutralized 79% of tested Tier 2 viruses (11/14), including four viruses that were not neutralized by VRC38.01 (TRO.11, CH119.10, 25710.2.43, and CE1176.A3) (Cale et al., 2017) (FIG. 3D, FIG. 9B). Of note, 3602-870 neutralized BG505 and ZM197, both of which were used as probes in the antigen screening library (FIG. 3D). 3602-870 bound BG505 DS-SOSIP by SPR and competed for BG505 DS-SOSIP binding to the greatest extent with VRC01 Fab (FIG. 3E). In summary, LIBRA-seq enabled the high-throughput, highly multiplexed screening of single B cells against many HIV antigen variants. This resulted in the identification of hundreds of antigen-specific monoclonal antibody leads from donor N90, with high-resolution antigen specificity mapping helping to facilitate rapid lead prioritization to identify a novel bNAb lineage.

### Example 5. Discussion

Disclosed herein is a method to interrogate antibody-antigen interactions via a sequencing-based readout were disclosed. New members of two known HIV-specific bNAb lineages were identified from previously characterized human infection samples and a novel bNAb lineage. Additionally, many other broadly-reactive HIV-specific antibodies were identified and investigated regarding their specificity for a subset of them. Within both HIV-1 infection samples, influenza-specific antibodies were also isolated using hemagglutinin screening probes, highlighting LIBRA-seq for use in methods of simultaneously screening B cell repertoires against multiple, diverse antigen targets. The NGS-based coupling of antibody sequence and specificity enables screening of potentially millions of single B cells for reactivity to a larger repertoire of epitopes than purely fluorescence-based methods, since sequence space is not hindered by spectral overlap. Using LIBRA-seq therefore helps to maximize lead discovery per experiment, an important consideration when preserving limited sample.

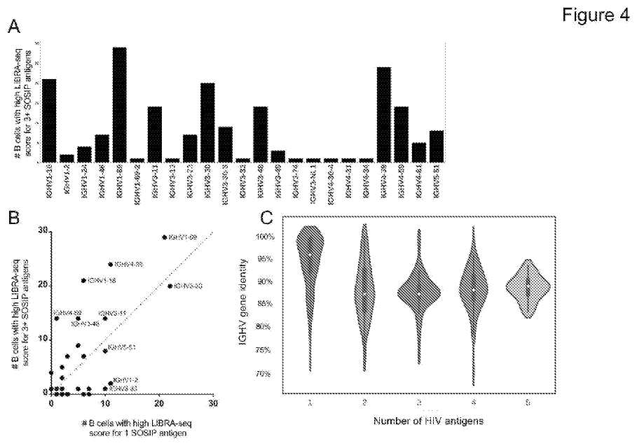

Beyond LIBRA-seq's importance in antibody discovery, the high-throughput coupling of antibody sequence and specificity can enable high-resolution immune profiling. For example, in donor N90, the use of specific germline genes (e.g., IGHV1-69, IGHV4-39, and IGHV1-18) was enriched in B cells that exhibited broad, as opposed to strain-specific, HIV-1 antigen reactivity (FIG. 4A-4B). In addition, an increase in somatic hypermutation levels was observed between B cells that bind a single SOSIP probe versus those that bind multiple probes (FIG. 4C). The elucidation of such relationships, enabled by the LIBRA-seq technology, can allow germline-targeting vaccine design efforts (Dosenovic et al., 2019; Jardine et al., 2013, 2016; Stamatatos et al., 2017) and can also allow the determination of the requirements for the acquisition of HIV-1 antigen cross-reactivity.

### Example 6. Methods and Materials

Antigen expression and purification. For the different LIBRA-seq experiments, a total of six HIV-1 gp140 SOSIP variants from strains BG505 (clade A), CZA97 (clade C), B41 (clade B), ZM197 (clade C), ZM106.9 (clade C), KNH1144 (clade A) and four influenza hemagglutinin variants from strains A/New Caledonia/20/99 (H1N1) (GenBank ACF41878), A/Michigan/45/2015 (H1N1) (GenBank AMA11475), A/Indonesia/5/2005 (H5N1) (GenBank ABP51969), and A/Anhui/1/2013 (H7N9) (GISAID EPI439507) were expressed as recombinant soluble antigens.

The single-chain variants (Georgiev et al., 2015) of BG505, CZA97, B41, ZM197, ZM106.9, and KNH1144 each containing an Avi tag, were expressed in 293F mammalian cells using polyethylenimine (PEI) transfection reagent and cultured for 5-7 days. Next, cultures were centrifuged at 6000 rpm for 20 minutes. Supernatant was 0.45 μm filtered with Nalgene Rapid Flow Disposable Filter Units with PES membrane, and then run slowly over an affinity column of agarose bound Galanthus nivalis lectin (Vector Laboratories cat no. AL-1243-5) at 4° C. The column was washed with PBS, and proteins were eluted with 30 mL of 1 M methyl-α-D-mannopyranoside. The protein elution was buffer exchanged 3× into PBS and concentrated using 30 kDa Amicon Ultra centrifugal filter units. Concentrated protein was run on a Superdex 200 Increase 10/300 GL sizing column on the AKTA FPLC system, and fractions were collected on an F9-R fraction collector. Fractions corresponding to correctly folded antigen were analyzed by SDS-PAGE, and antigenicity by ELISA was characterized with known monoclonal antibodies specific for that antigen.

Recombinant HA proteins all contained the HA ectodomain with a point mutation at the sialic acid-binding site (Y98F), T4 fibritin foldon trimerization domain, Avi tag, and hexahistidine tag, and were expressed in Expi 293F mammalian cells using Expifectamine 293 transfection reagent (Thermo Fisher Scientific) cultured for 4-5 days. Culture supernatant was harvested and cleared as above, and then adjusted pH and NaCl concentration by adding 1M Tris-HCl (pH 7.5) and 5M NaCl to 50 mM and 500 mM, respectively. Ni Sepharose excel resin (GE Healthcare) was added to the supernatant to capture hexahistidine tag. Resin was separated on a column by gravity and captured HA protein was eluted by a Tris-NaCl (pH 7.5) buffer containing 300 mM imidazole. The eluate was further purified by a size exclusion chromatography with a HiLoad 16/60 Superdex 200 column (GE Healthcare). Fractions containing HA were concentrated, analyzed by SDS-PAGE and tested for antigenicity by ELISA with known antibodies. Proteins were frozen in LN2 and stored at −80C° until use.

All antigens included an AviTag modification at the C-terminus of their sequence, and after purification, each AviTag labeled antigen was biotinylated using the BirA-500: BirA biotin-protein ligase standard reaction kit (Avidity LLC, cat no. BirA500).

Oligonucleotide barcode design. Oligo used herein possess a 13-15 bp antigen barcode, a sequence capable of annealing to the template switch oligo that is part of the 10× bead-delivered oligos, and contain truncated TruSeq small RNA read 1 sequences in the following structure: 5′-CCTTGGCACCCGAGAATTCCANNNNNNNNNNNNNCCCATATAAGA*A*A-3′ (SEQ ID NO: 949), where Ns represent the antigen barcode. For the cell line and NIAID45 experiments, we used the following antigen barcodes: CATGATTGGCTCA (SEQ ID NO: 950) (BG505), TGTCCGGCAATAA (SEQ ID NO: 951) (CZA97), GATCGTAATACCA (SEQ ID NO: 952) (H1 A/New Caledonia/20/99). For the N90 experiment, we used longer antigen barcodes (15 bp), as follows: TCCTTTCCTGATAGG (SEQ ID NO: 953) (ZM106.9), TAACTCAGGGCCTAT (SEQ ID NO: 954) (KNH1144), GCTCCTTTACACGTA (SEQ ID NO: 955) (ZM197), GCAGCGTATAAGTCA (SEQ ID NO: 956) (B41), ATCGTCGAGAGCTAG (SEQ ID NO: 957) (BG505), CAGGTCCCTTATTTC (SEQ ID NO: 958) (A/Indonesia/5/2005), ACAATTTGTCTGCGA (SEQ ID NO: 959) (A/Anhui/1/2013), TGACCTTCCTCTCCT (SEQ ID NO: 960) (A/Michigan/45/2015), AATCACGGTCCTTGT (SEQ ID NO: 961) (A/New Caledonia/20/99). Oligos were ordered from Sigma-Aldrich and IDT with a 5′ amino modification and HPLC purified.

Conjugation of oligonucleotide barcodes to antigens. For each antigen, a unique DNA “barcode” was directly conjugated to the antigen itself. In particular, 5′ amino-oligonucleotides were conjugated directly to each antigen using the Solulink Protein-Oligonucleotide Conjugation Kit (TriLink cat no. S-9011) according to manufacturer's instructions. Briefly, the oligo and protein were desalted, and then the amino-oligo was modified with the 4FB crosslinker, and the biotinylated antigen protein was modified with S-HyNic. Then, the 4FB-oligo and the HyNic-antigen were mixed together. This causes a stable bond to form between the protein and the oligonucleotide. The concentration of the antigen-oligo conjugates was determined by a BCA assay, and the HyNic molar substitution ratio of the antigen-oligo conjugates was analyzed using the NanoDrop according to the Solulink protocol guidelines. AKTA FPLC was used to remove excess oligonucleotide from the protein-oligo conjugates. Additionally, the antigen-oligo conjugates were analyzed via SDS-PAGE with a silver stain.

Fluorescent labeling of antigens. After attaching DNA barcodes directly to a biotinylated antigen, the barcoded antigens were mixed with streptavidin labeled with fluorophore phycoerythrin (PE). The streptavidin-PE was mixed with biotinylated antigen at a 5× molar excess of antigen to streptavidin. 1/5 of the streptavidin-oligo conjugate was added to the antigen every 20 minutes with constant rotation at 4° C.

B cell lines production and identification by sequencing. B cell lines were engineered from a clone of Ramos Burkitt's lymphoma that do not display endogenous antibody, and they ectopically express specific surface IgM B cell receptor sequences. The B cell lines used expressed B cell receptor sequences for HIV-1 specific antibody VRC01 and influenza specific antibody Fe53. The cells are cultured at 37° C. with 5% CO2 saturation in complete RPMI, made up of RPMI supplemented with 15% fetal bovine serum, 1% L-Glutamine, and 1% Penicillin/Streptomycin. Although endogenous heavy chains are scrambled, endogenous light chain transcripts remain and are detectable by sequencing. We thus identified and classified single Ramos Burkitt's B cells as either VRC01 or FE53 based on their heavy chain sequences. These Ramos B cell lines were validated for binding to our antigen probes by FACS.

Donor PBMCs. Donor NIAID45 Peripheral blood mononuclear cells were collected from donor NIAID45 on July 12, 2007. Donor NIAID45, from whom antibodies VRC01, VRC02, VRC03, VRC06, VRC07, VRC08, NIH45-46, and others from the VRC01 bNAb lineage had been previously isolated, was enrolled in investigational review board approved clinical protocols at the National Institute of Allergy and Infectious Diseases and had been living with HIV without antiretroviral treatment for approximately 17 years at the time of sample collection. Donor N90 Peripheral blood mononuclear cells were collected from donor N90 on May 29, 2008. Donor N90, from whom antibody lineage VRC38 had been previously isolated, was enrolled in investigational review board approved clinical protocols at the National Institute of Allergy and Infectious Diseases and had been living with HIV without antiretroviral treatment through the timepoint of sample collection since diagnosis in 1985 (Wu et al., 2012).

Enrichment of antigen-specific IgG+B cells. For the given sample, cells were stained and mixed with fluorescently labeled DNA-barcoded antigens and other antibodies, and then sorted using fluorescence activated cell sorting (FACS). First, cells were counted and viability was assessed using Trypan Blue. Then, cells were washed with DPBS supplemented with 1% Bovine serum albumin (BSA) through centrifugation at 300 g for 7 minutes. Cells were resuspended in PBS-BSA and stained with a variety of cell markers. For donor NIAID 45 PBMCs, these markers included CD3-APCCy7, IgG-FITC, CD19-BV711, CD14-V500, and LiveDead-V500. Additionally, fluorescently labeled antigen-oligo conjugates (described above) were added to the stain, so antigen-specific sorting could occur. For donor N90 PBMCs, these markers included LiveDead-APCCy7, CD14-APCCy7, CD3-FITC, CD19-BV711, and IgG-PECy5. Additionally, fluorescently labeled antigen-oligo conjugates were added to the stain, so antigen-specific sorting could occur. After staining in the dark for 30 minutes at room temperature, cells were washed 3 times with PBS-BSA at 300 g for 7 minutes. Then, cells were resuspended in PBS-BSA and sorted on the cell sorter. Antigen positive cells were bulk sorted and then they were delivered to the Vanderbilt VANTAGE sequencing core at an appropriate target concentration for 10× Genomics library preparation and NGS analysis. FACS data were analyzed using Cytobank (Kotecha et al., 2010).

10× single cell processing and next generation sequencing. Single-cell suspensions were loaded onto the Chromium microfluidics device (10× Genomics) and processed using the B-cell VDJ solution according to manufacturer's suggestions for a target capture of 10,000 B cells per 1/8 10× cassette for B cell lines, 9,000 cells for B cells from donor NIAID45, and 4,000 for donor N90, with minor modifications in order to intercept, amplify and purify the antigen barcode libraries. The library preparation follows the CITE-seq protocol (available at cite-seq.com), with the exception of an increase in the number of PCR cycles of the antigen barcodes. Briefly, following cDNA amplification using an additive primer (5′ -CCTTGGCACCCGAGAATT*C*C-3′) (SEQ ID NO: 962) to increase the yield of antigen barcode libraries (Stoeckius et al., 2017), SPRI separation was used to size separate antigen barcode libraries from cellular mRNA libraries, PCR amplified for 10-12 cycles, and purified using 1.6× purification. Sample preparation for the cellular mRNA library continued according to 10× Genomics-suggested protocols, resulting in Illumina-ready libraries. Following library construction, we sequenced both BCR and antigen barcode libraries on a NovaSeq 6000 at the VANTAGE sequencing core, dedicating ˜2.5% of a flow cell to each experiment, with a target 10% of this fraction dedicated to antigen barcode libraries. This resulted in ˜334 5 million reads for the cell line V(D)J libraries (˜96,500 reads/cell), ˜376.3 million reads for donor NIAID45 V(D)J libraries (˜79,300 reads/cell), and ˜272 4 million reads for the N90 V(D)J libraries (˜151,400 reads/cell). Additionally, this sequencing depth resulted in ˜46.7 million total reads for antigen barcode library of the cell lines, ˜39 6 million reads for the antigen barcode library of donor NIAID45, and ˜82 9 million reads for the antigen barcode library for N90.

Processing of antigen barcode reads and BCR sequence contigs. A pipeline shown herein takes paired-end fastq files of oligo libraries as input, processes and annotates reads for cell barcode, UMI, and antigen barcode, and generates a cell barcode—antigen barcode UMI count matrix. BCR contigs are processed using cellranger (10× Genomics) using GRCh38 as reference. For the antigen barcode libraries, initial quality and length filtering is carried out by fastp (Chen et al., 2018) using default parameters for filtering. This results in only high-quality reads being retained in the antigen barcode library (FIG. 11). In a histogram of insert lengths, this results in a sharp peak of the expected insert size of 52-54 (FIG. 9B-9C). Fastx_collapser is then used to group identical sequences and convert the output to deduplicated fasta files. Then, having removed low-quality reads, just the R2 sequences were processed, as the entire insert is present in both R1 and R2. Each unique R2 sequence (or R1, or the consensus of R1 and R2) was processed one by one using the following steps: (1) The reverse complement of the R2 sequence was determined (Skip step 1 if using R1). (2) The sequence was screened for possessing an exact match to any of the valid 10× cell barcodes present in the filtered_contig.fasta file output by cell ranger during processing of BCR V(D)J fastq files. Sequences without a BCR-associated cell barcode were discarded. (3) The 10 bases immediate 3′ to the cell barcode were annotated as the read's UMI. (4) The remainder of the sequence 3′ to the UMI is screened for a 13 or 15 bp sequence with a hamming distance of 0, 1, or 2 to any of the antigen barcodes used in the screening library. Following this processing, only sequences with lengths of 51 to 58 were retained, thus allowing for a deletion, an insertion outside the cell barcode, or bases flanking the cell barcode. This general process requires that sequences possess all elements needed for analysis (cell barcode, UMI, and antigen barcode), but is permissive to insertions or deletions in the TSO region between the UMI and antigen barcode. After processing each sequence one-by-one, we screened for cell barcode—UMI—antigen barcode collisions. Any cell barcode—UMI combination (indicative of a unique oligo molecule) that had multiple antigen barcodes associated with it was removed. A cell barcode—antigen barcode UMI count matrix was then constructed, which served as the basis of subsequent analysis. Additionally, the BCR contigs were aligned (filtered_contigs.fasta file output by Cellranger, 10× Genomics) to IMGT reference genes using HighV-Quest (Alamyar et al., 2012). The output of HighV-Quest is parsed using ChangeO (Gupta et al., 2015), and merged with the UMI count matrix.

Determination of LIBRA-seq Score. Starting with the UMI count matrix, all counts of 1, 2, or 3 UMIs were set to 0, with the idea that these low counts can be attributed to noise. After this, the UMI count matrix was subset to contain only cells with a count of at least 4 UMIs for at least 1 antigen. The centered-log ratios (CLR) of each antigen UMI count for each cell were then calculated (Mimitou et al., 2019; Stoeckius et al., 2017, 2018). Because UMI counts were on different scales for each antigen, due to differential oligo loading during oligo-antigen conjugation, the CLRs UMI counts were rescaled using the StandardScaler method in scikit learn (Pedregosa and Varoquaux, 2011). Lastly, A correction procedure was performed to the z-score-normalized CLRs from UMI counts of 0, setting them to the minimum for each antigen for donor NIAID 45 and N90 experiments, and to −1 for the Ramos B cell line experiment. These CLR-transformed, Z-score-normalized, corrected values served as the final LIBRA-seq scores. LIBRA-seq scores were visualized using Cytobank (Kotecha et al., 2010).

Phylogenetic trees. Phylogenetic trees of antibody heavy chain sequences were constructed in order to assess the relative relatedness of antibodies within a given lineage. For the VRC01 lineage, the 29 sequences identified by LIBRA-seq and 52 sequences identified from the literature were aligned using clustal within Geneious. We then used the PhyML maximum likelihood (Guindon et al., 2009) plugin in Geneious (available at www.geneious.com/plugins/phyml-plugin/) to infer a phylogenetic tree. The resulting tree was then rooted to the inferred unmutated common ancestor (Bonsignori et al., 2018) (accession MK032222). A similar process was used to build a phylogenetic tree for lineage 2121, with one exception. Rather than using an inferred germline precursor, the IGHV and IGHJ genes were germline-reverted and the CDRH3 nucleotide sequence of the lineage member was used with the least IGHV somatic mutation. Trees were annotated and visualized in iTol (Letunic and Bork, 2019).

Antibody expression and purification. For each antibody, variable genes were inserted into plasmids encoding the constant region for the heavy chain (pFUSE-CHIg, Invivogen) and light chain (pFUSE2-CLIg, Invivogen) and synthesized from GenScript. In cases where the IgBLAST-aligned sequence was missing any residues at the beginning of framework 1 or end of framework 4, sequences were completed with germline residues. mAbs were expressed in Expi 293F mammalian cells by co-transfecting heavy chain and light chain expressing plasmids using polyethylenimine (PEI) transfection reagent and cultured for 5-7 days. Next, cultures were centrifuged at 6000 rpm for 20 minutes. Supernatant was 0.45 μm filtered with Nalgene Rapid Flow Disposable Filter Units with PES membrane. Filtered supernatant was run over a column containing Protein A agarose resin that had been equilibrated with PBS. The column was washed with PBS, and then antibodies were eluted with 100 mM Glycine HCl at pH 2.7 directly into a 1:10 volume of 1 M Tris-HCL pH 8. Eluted antibodies were buffer exchanged into PBS 3 times using 10 kDa Amicon Ultra centrifugal filter units.

Enzyme linked immunosorbent assay (ELISA). For ELISAs, soluble hemagglutinin protein was plated at 2 μg/ml overnight at 4° C. The next day, plates were washed three times with PBS supplemented with 0.05% Tween20 (PBS-T) and coated with 5% milk powder in PBS-T. Plates were incubated for one hour at room temperature and then washed three times with PBS-T. Primary antibodies were diluted in 1% milk in PBS-T, starting at 10 μg/ml with a serial 1:5 dilution and then added to the plate. The plates were incubated at room temperature for one hour and then washed three times in PBS-T. The secondary antibody, goat anti-human IgG conjugated to peroxidase, was added at 1:20,000 dilution in 1% milk in PBS-T to the plates, which were incubated for one hour at room temperature. Plates were washed three times with PBS-T and then developed by adding TMB substrate to each well. The plates were incubated at room temperature for ten minutes, and then 1 N sulfuric acid was added to stop the reaction. Plates were read at 450 nm.

For recombinant trimer capture for single-chain SOSIPs, 2 μg/ml of a mouse anti-AviTag antibody (GenScript) was coated overnight at 4 C in phosphate-buffered saline (PBS) (pH 7.5). The next day plates were washed three times with PBS-T, and blocked with 5% milk in PBS-T. After an hour incubation at room temperature and three washes with PBS-T, 2 μg/ml of recombinant trimer proteins diluted in 1% milk PBS-T were added to the plate and incubated for one hour at room temperature. Primary and secondary antibodies, along with substrate and sulfuric acid, were added as described above. ELISAs were performed in at least two experimental replicates and data were graphed using GraphPad Prism 8.0.0. Data shown is representative of one replicate, with error bars representing standard error of the mean for technical duplicates within that experiment. The area under the curve (AUC) was calculated using GraphPad Prism 8.0.0.

TZM-bl Neutralization Assays. Antibody neutralization was assessed using the TZM-bl assay as described (Sarzotti-Kelsoe et al., 2014). This standardized assay measures antibody-mediated inhibition of infection of JC53BL-13 cells (also known as TZM-bl cells) by molecularly cloned Env-pseudoviruses. Viruses that are highly sensitive to neutralization (Tier 1) and those representing circulating strains that are moderately sensitive (Tier 2) were included. Antibodies were tested against a variety of Tier 1 viruses and the Tier 2 Global panel plus additional viruses, including a subset of the antigens used for LIBRA-seq. Murine leukemia virus (MLV) was included as an HIV-specificity control and VRC01 was used as a positive control. Results are presented as the concentration of monoclonal antibody (in μg/ml) required to inhibit 50% of virus infection (IC₅₀).

Surface Plasmon Resonance and Fab competition. The binding of antibody 2723-2121 to BG505 DS-SOSIP (Do Kwon et al., 2015) was assessed by surface plasmon resonance on Biacore T-200 (GE-Healthcare) at 25° C. with HBS-EP+ (10 mM HEPES, pH 7.4, 150 mM NaCl, 3 mM EDTA, and 0.05% surfactant P-20) as the running buffer. Antibodies VRC01 and PGT145 were tested as positive control, and antibody 17b was tested as negative control to confirm that the trimer was in the closed conformation. Antibody 2723-2121 was captured on a flow cell of CM5 chip immobilized with ˜7500 RU of anti-human Fc antibody, and binding was measured by flowing over a 200 nM solution BG505-DS SOSIP in running buffer. Similar runs were performed with VRC01, PGT145 and 17b IgGs. To determine the epitope of antibody 2723-2121, we captured 2723-2121 IgG on a single flow cell of CM5 chip immobilized with ˜7500 RU of anti-human Fc antibody. Next 200 nM BG505 DS-SOSIP, either alone or with different concentrations of antigen binding fragments (Fab) of VRC01 or PGT145 or VRC34 was flowed over the captured 2723-2121 flow cell for 60 s at a rate of 10 μl/min. The surface was regenerated between injections by flowing over 3M MgCl₂ solution for 10 s with flow rate of 100 μl/min. Blank sensorgrams were obtained by injection of same volume of HBS-EP+ buffer in place of trimer with Fabs solutions. Sensorgrams of the concentration series were corrected with corresponding blank curves. The binding of antibody 3602-870 to BG505 DS-SOSIP was assessed by surface plasmon resonance in the same way as described for 2723-2121. For 3602-870, competition experiments were performed with PGT145 Fab, PGT122 Fab, and VRC01 Fab.

ADCP, ADCD, Trogocytosis, ADCC Assays. Antibody-dependent cellular phagocytosis (ADCP) was performed using gp120 ConC coated neutravidin beads as previously described (Ackerman et at, 2011). Phagocytosis score was determined as the percentage of cells that took up beads multiplied by the fluorescent intensity of the beads. Antibody-dependent complement deposition (ADCD) was performed as in (Richardson et al., 2018a) where CEM.NKR.CCRS gp120 ConC coated target cells were opsonized with mAb and incubated with complement from a healthy donor. C3b deposition was then determined by flow cytometry with complement deposition score determined as the percentage of C3b positive cells multiplied by the fluorescence intensity. Antibody dependent cellular trogocytosis (ADCT) was measured as the percentage transfer of PKH26 dye of the surface of CEM.NKR.CCRS target cells to CSFE stained monocytic cell line THP-1 cells in the presence of HIV specific mAbs as described elsewhere (Richardson et al., 2018b). Antibody-dependent cellular cytotoxicity (ADCC) was done using a GranToxiLux based assay (Pollara et al., 2011) with gp120 ConC coated CEM.NKR.CCRS target cells and PBMCs from a healthy donor. The percentage of granzyme B present in target cells was measured by flow cytometry.

Statistics. ELISA error bars (standard error) were calculated using GraphPad Prism version 8.0.0. The Pearson's r value comparing BG505 and CZA97 LIBRA-seq scores for Ramos B-cell lines was calculated using Cytobank. Spearman correlations and associated p values were calculated using SciPy in Python.

TABLE 1
Nucleic acid sequences encoding heavy and light chains of antibodies and the cell barcodes thereof.

| | | | SEQ ID NO for | SEQ ID NO for | |
|---|---|---|---|---|---|
| | | SEQ ID NO for | Heavy Chain | Light Chain | |
| Donor | Index | Cell Barcode | Contig | Contig | Selection logic |
| N90 | 585 | 1 | 223 | 445 | Cross-reactive HIV |
| N90 | 1758 | 2 | 224 | 446 | Cross-reactive HIV |
| N90 | 3086 | 3 | 225 | 447 | Cross-reactive HIV |
| N90 | 2163 | 4 | 226 | 448 | Cross-reactive HIV |
| N90 | 627 | 5 | 227 | 449 | Cross-reactive HIV |
| N90 | 3218 | 6 | 228 | 450 | Cross-reactive HIV |
| N90 | 490 | 7 | 229 | 451 | Cross-reactive HIV |
| N90 | 84 | 8 | 230 | 452 | Cross-reactive HIV |
| N90 | 3023 | 9 | 231 | 453 | Cross-reactive HIV |
| N90 | 370 | 10 | 232 | 454 | Cross-reactive HIV |
| N90 | 2064 | 11 | 233 | 455 | Cross-reactive HIV |
| N90 | 2673 | 12 | 234 | 456 | Cross-reactive HIV |
| N90 | 3279 | 13 | 235 | 457 | Cross-reactive HIV |
| N90 | 2394 | 14 | 236 | 458 | Cross-reactive HIV |
| N90 | 2429 | 15 | 237 | 459 | Cross-reactive HIV |
| N90 | 1582 | 16 | 238 | 460 | Cross-reactive HIV |
| N90 | 2808 | 17 | 239 | 461 | Cross-reactive HIV |
| N90 | 2320 | 18 | 240 | 462 | Cross-reactive HIV |
| N90 | 2052 | 19 | 241 | 463 | Cross-reactive HIV |
| N90 | 1057 | 20 | 242 | 464 | Cross-reactive HIV |
| N90 | 1140 | 21 | 243 | 465 | Cross-reactive HIV |
| N90 | 2538 | 22 | 244 | 466 | Cross-reactive HIV |
| N90 | 2212 | 23 | 245 | 467 | Cross-reactive HIV |
| N90 | 1925 | 24 | 246 | 468 | Cross-reactive HIV |
| N90 | 528 | 25 | 247 | 469 | Cross-reactive HIV |
| N90 | 3353 | 26 | 248 | 470 | Cross-reactive HIV |
| N90 | 2302 | 27 | 249 | 471 | Cross-reactive HIV |
| N90 | 318 | 28 | 250 | 472 | Cross-reactive HIV |
| N90 | 3258 | 29 | 251 | 473 | Cross-reactive HIV |
| N90 | 2664 | 30 | 252 | 474 | Cross-reactive HIV |
| N90 | 2548 | 31 | 253 | 475 | Cross-reactive HIV |
| N90 | 1762 | 32 | 254 | 476 | Cross-reactive HIV |
| N90 | 1062 | 33 | 255 | 477 | Cross-reactive HIV |
| N90 | 1284 | 34 | 256 | 478 | Cross-reactive HIV |
| N90 | 592 | 35 | 257 | 479 | Cross-reactive HIV |
| N90 | 2876 | 36 | 258 | 480 | Cross-reactive HIV |
| N90 | 1887 | 37 | 259 | 481 | Cross-reactive HIV |
| N90 | 1178 | 38 | 260 | 482 | Cross-reactive HIV |
| N90 | 2507 | 39 | 261 | 483 | Cross-reactive HIV |
| N90 | 957 | 40 | 262 | 484 | Cross-reactive HIV |
| N90 | 3359 | 41 | 263 | 485 | Cross-reactive HIV |
| N90 | 1904 | 42 | 264 | 486 | Cross-reactive HIV |
| N90 | 1692 | 43 | 265 | 487 | Cross-reactive HIV |
| N90 | 1661 | 44 | 266 | 488 | Cross-reactive HIV |
| N90 | 1407 | 45 | 267 | 489 | Cross-reactive HIV |
| N90 | 1042 | 46 | 268 | 490 | Cross-reactive HIV |
| N90 | 1954 | 47 | 269 | 491 | Cross-reactive HIV |
| N90 | 1442 | 48 | 270 | 492 | Cross-reactive HIV |
| N90 | 2211 | 49 | 271 | 493 | Cross-reactive HIV |
| N90 | 451 | 50 | 272 | 494 | Cross-reactive HIV |
| N90 | 3544 | 51 | 273 | 495 | Cross-reactive HIV |
| N90 | 3232 | 52 | 274 | 496 | Cross-reactive HIV |
| N90 | 3226 | 53 | 275 | 497 | Cross-reactive HIV |
| N90 | 2985 | 54 | 276 | 498 | Cross-reactive HIV |
| N90 | 180 | 55 | 277 | 499 | Cross-reactive HIV |
| N90 | 2427 | 56 | 278 | 500 | Cross-reactive HIV |
| N90 | 1433 | 57 | 279 | 501 | Cross-reactive HIV |
| N90 | 979 | 58 | 280 | 502 | Cross-reactive HIV |
| N90 | 889 | 59 | 281 | 503 | Cross-reactive HIV |
| N90 | 442 | 60 | 282 | 504 | Cross-reactive HIV |
| N90 | 389 | 61 | 283 | 505 | Cross-reactive HIV |
| N90 | 3494 | 62 | 284 | 506 | Cross-reactive HIV |
| N90 | 3093 | 63 | 285 | 507 | Cross-reactive HIV |
| N90 | 2420 | 64 | 286 | 508 | Cross-reactive HIV |
| N90 | 2232 | 65 | 287 | 509 | Cross-reactive HIV |
| N90 | 1884 | 66 | 288 | 510 | Cross-reactive HIV |
| N90 | 463 | 67 | 289 | 511 | Cross-reactive HIV |
| N90 | 334 | 68 | 290 | 512 | Cross-reactive HIV |
| N90 | 223 | 69 | 291 | 513 | Cross-reactive HIV |
| N90 | 3415 | 70 | 292 | 514 | Cross-reactive HIV |
| N90 | 1992 | 71 | 293 | 515 | Cross-reactive HIV |
| N90 | 1987 | 72 | 294 | 516 | Cross-reactive HIV |
| N90 | 1977 | 73 | 295 | 517 | Cross-reactive HIV |
| N90 | 1848 | 74 | 296 | 518 | Cross-reactive HIV |
| N90 | 1728 | 75 | 297 | 519 | Cross-reactive HIV |
| N90 | 1567 | 76 | 298 | 520 | Cross-reactive HIV |
| N90 | 1506 | 77 | 299 | 521 | Cross-reactive HIV |
| N90 | 1416 | 78 | 300 | 522 | Cross-reactive HIV |
| N90 | 1027 | 79 | 301 | 523 | Cross-reactive HIV |
| N90 | 934 | 80 | 302 | 524 | Cross-reactive HIV |
| N90 | 652 | 81 | 303 | 525 | Cross-reactive HIV |
| N90 | 624 | 82 | 304 | 526 | Cross-reactive HIV |
| N90 | 431 | 83 | 305 | 527 | Cross-reactive HIV |
| N90 | 350 | 84 | 306 | 528 | Cross-reactive HIV |
| N90 | 3345 | 85 | 307 | 529 | Cross-reactive HIV |
| N90 | 2504 | 86 | 308 | 530 | Cross-reactive HIV |
| N90 | 1753 | 87 | 309 | 531 | Cross-reactive HIV |
| N90 | 1690 | 88 | 310 | 532 | Cross-reactive HIV |
| N90 | 1324 | 89 | 311 | 533 | Cross-reactive HIV |
| N90 | 1314 | 90 | 312 | 534 | Cross-reactive HIV |
| N90 | 155 | 91 | 313 | 535 | Cross-reactive HIV |
| N90 | 1866 | 92 | 314 | 536 | Cross-reactive HIV |
| N90 | 654 | 93 | 315 | 537 | Cross-reactive HIV |
| N90 | 1487 | 94 | 316 | 538 | Cross-reactive HIV |
| N90 | 842 | 95 | 317 | 539 | Cross-reactive HIV |
| N90 | 523 | 96 | 318 | 540 | Cross-reactive HIV |
| N90 | 284 | 97 | 319 | 541 | Cross-reactive HIV |
| N90 | 208 | 98 | 320 | 542 | Cross-reactive HIV |
| N90 | 1149 | 99 | 321 | 543 | Cross-reactive HIV |
| N90 | 1882 | 100 | 322 | 544 | Cross-reactive HIV |
| N90 | 1662 | 101 | 323 | 545 | Cross-reactive HIV |
| N90 | 1572 | 102 | 324 | 546 | Cross-reactive HIV |
| N90 | 404 | 103 | 325 | 547 | Cross-reactive HIV |
| N90 | 2978 | 104 | 326 | 548 | Cross-reactive HIV |
| N90 | 1261 | 105 | 327 | 549 | Cross-reactive HIV |
| N90 | 845 | 106 | 328 | 550 | Cross-reactive HIV |
| N90 | 1125 | 107 | 329 | 551 | Cross-reactive HIV |
| N90 | 3035 | 108 | 330 | 552 | Cross-reactive HIV |
| N90 | 3272 | 109 | 331 | 553 | Cross-reactive HIV |
| N90 | 2759 | 110 | 332 | 554 | Cross-reactive HIV |
| N90 | 2638 | 111 | 333 | 555 | Cross-reactive HIV |
| N90 | 2014 | 112 | 334 | 556 | Cross-reactive HIV |
| N90 | 1824 | 113 | 335 | 557 | Cross-reactive HIV |
| N90 | 1612 | 114 | 336 | 558 | Cross-reactive HIV |
| N90 | 1478 | 115 | 337 | 559 | Cross-reactive HIV |
| N90 | 1422 | 116 | 338 | 560 | Cross-reactive HIV |
| N90 | 942 | 117 | 339 | 561 | Cross-reactive HIV |
| N90 | 818 | 118 | 340 | 562 | Cross-reactive HIV |
| N90 | 445 | 119 | 341 | 563 | Cross-reactive HIV |
| N90 | 183 | 120 | 342 | 564 | Cross-reactive HIV |
| N90 | 30 | 121 | 343 | 565 | Cross-reactive HIV |
| N90 | 29 | 122 | 344 | 566 | Cross-reactive HIV |
| N90 | 3477 | 123 | 345 | 567 | Cross-reactive HIV |
| N90 | 2845 | 124 | 346 | 568 | Cross-reactive HIV |
| N90 | 587 | 125 | 347 | 569 | Cross-reactive HIV |
| N90 | 3330 | 126 | 348 | 570 | Cross-reactive HIV |
| N90 | 3047 | 127 | 349 | 571 | Cross-reactive HIV |
| N90 | 2612 | 128 | 350 | 572 | Cross-reactive HIV |
| N90 | 2148 | 129 | 351 | 573 | Cross-reactive HIV |
| N90 | 1657 | 130 | 352 | 574 | Cross-reactive HIV |
| N90 | 1016 | 131 | 353 | 575 | Cross-reactive HIV |
| N90 | 968 | 132 | 354 | 576 | Cross-reactive HIV |
| N90 | 277 | 133 | 355 | 577 | Cross-reactive HIV |
| N90 | 2309 | 134 | 356 | 578 | Cross-reactive HIV |
| N90 | 3140 | 135 | 357 | 579 | Cross-reactive HIV |
| N90 | 2790 | 136 | 358 | 580 | Cross-reactive HIV |
| N90 | 2726 | 137 | 359 | 581 | Cross-reactive HIV |
| N90 | 1308 | 138 | 360 | 582 | Cross-reactive HIV |
| N90 | 991 | 139 | 361 | 583 | Cross-reactive HIV |
| N90 | 406 | 140 | 362 | 584 | Cross-reactive HIV |
| N90 | 137 | 141 | 363 | 585 | Cross-reactive HIV |
| N90 | 3005 | 142 | 364 | 586 | Cross-reactive HIV |
| N90 | 2745 | 143 | 365 | 587 | Cross-reactive HIV |
| N90 | 3439 | 144 | 366 | 588 | Cross-reactive HIV |
| N90 | 3400 | 145 | 367 | 589 | Cross-reactive HIV |
| N90 | 1921 | 146 | 368 | 590 | Cross-reactive HIV |
| N90 | 1126 | 147 | 369 | 591 | Cross-reactive HIV |
| N90 | 256 | 148 | 370 | 592 | Cross-reactive HIV |
| N90 | 3109 | 149 | 371 | 593 | Cross-reactive HIV |
| N90 | 2967 | 150 | 372 | 594 | Cross-reactive HIV |
| N90 | 2337 | 151 | 373 | 595 | Cross-reactive HIV |
| N90 | 1705 | 152 | 374 | 596 | Cross-reactive HIV |
| N90 | 492 | 153 | 375 | 597 | Cross-reactive HIV |
| N90 | 1479 | 154 | 376 | 598 | Cross-reactive HIV |
| N90 | 2002 | 155 | 377 | 599 | Cross-reactive HIV |
| N90 | 1813 | 156 | 378 | 600 | Cross-reactive HIV |
| N90 | 1048 | 157 | 379 | 601 | Cross-reactive HIV |
| N90 | 931 | 158 | 380 | 602 | Cross-reactive HIV |
| N90 | 460 | 159 | 381 | 603 | Cross-reactive HIV |
| N90 | 245 | 160 | 382 | 604 | Cross-reactive HIV |
| N90 | 3543 | 161 | 383 | 605 | Cross-reactive HIV |
| N90 | 2495 | 162 | 384 | 606 | Cross-reactive HIV |
| N90 | 2294 | 163 | 385 | 607 | Cross-reactive HIV |
| N90 | 91 | 164 | 386 | 608 | Cross-reactive HIV |
| N90 | 2379 | 165 | 387 | 609 | Cross-reactive HIV |
| N90 | 1851 | 166 | 388 | 610 | Cross-reactive HIV |
| N90 | 1357 | 167 | 389 | 611 | Cross-reactive HIV |
| N90 | 129 | 168 | 390 | 612 | Cross-reactive HIV |
| N90 | 48 | 169 | 391 | 613 | Cross-reactive HIV |
| N90 | 1287 | 170 | 392 | 614 | Cross-reactive HIV |
| N90 | 505 | 171 | 393 | 615 | Cross-reactive HIV |
| N90 | 3434 | 172 | 394 | 616 | Cross-reactive HIV |
| N90 | 3260 | 173 | 395 | 617 | Cross-reactive HIV |
| N90 | 51 | 174 | 396 | 618 | Cross-reactive HIV |
| N90 | 3441 | 175 | 397 | 619 | Cross-reactive HIV |
| N90 | 2535 | 176 | 398 | 620 | Cross-reactive HIV |
| N90 | 510 | 177 | 399 | 621 | Cross-reactive HIV |
| N90 | 328 | 178 | 400 | 622 | Cross-reactive HIV |
| N90 | 3497 | 179 | 401 | 623 | Cross-reactive HIV |
| N90 | 1549 | 180 | 402 | 624 | Cross-reactive HIV |
| N90 | 884 | 181 | 403 | 625 | Cross-reactive HIV |
| N90 | 2943 | 182 | 404 | 626 | Cross-reactive HIV |
| N90 | 2487 | 183 | 405 | 627 | Cross-reactive HIV |
| N90 | 1733 | 184 | 406 | 628 | Cross-reactive HIV |
| N90 | 3333 | 185 | 407 | 629 | Cross-reactive HIV |
| N90 | 3087 | 186 | 408 | 630 | Cross-reactive Flu |
| N90 | 1282 | 187 | 409 | 631 | Cross-reactive Flu |
| N90 | 2363 | 188 | 410 | 632 | Cross-reactive Flu |
| N90 | 251 | 189 | 411 | 633 | Cross-reactive Flu |
| N90 | 1849 | 190 | 412 | 634 | Cross-reactive Flu |
| N90 | 3139 | 191 | 413 | 635 | Cross-reactive Flu |
| N90 | 3455 | 192 | 414 | 636 | Cross-reactive Flu |
| N90 | 3180 | 193 | 415 | 637 | Cross-reactive Flu |
| N90 | 1993 | 194 | 416 | 638 | Cross-reactive Flu |
| N90 | 206 | 195 | 417 | 639 | Cross-reactive Flu |
| N90 | 2361 | 196 | 418 | 640 | Cross-reactive Flu |
| N90 | 218 | 197 | 419 | 641 | Cross-reactive Flu |
| N90 | 833 | 198 | 420 | 642 | Cross-reactive Flu |
| N90 | 2976 | 199 | 421 | 643 | Cross-reactive Flu |
| N90 | 2883 | 200 | 422 | 644 | Cross-reactive Flu |
| N90 | 1910 | 201 | 423 | 645 | Cross-reactive Flu |
| N90 | 1724 | 202 | 424 | 646 | Cross-reactive Flu |
| N90 | 377 | 203 | 425 | 647 | Cross-reactive Flu |
| N90 | 1757 | 204 | 426 | 648 | Cross-reactive Flu |
| N90 | 3326 | 205 | 427 | 649 | Cross-reactive Flu |
| N90 | 1864 | 206 | 428 | 650 | Cross-reactive Flu |
| N90 | 2822 | 207 | 429 | 651 | Cross-reactive Flu |
| N90 | 1373 | 208 | 430 | 652 | Cross-reactive Flu |
| N90 | 2709 | 209 | 431 | 653 | Cross-reactive Flu |
| N90 | 2496 | 210 | 432 | 654 | Cross-reactive Flu |
| N90 | 2018 | 211 | 433 | 655 | Cross-reactive Flu |
| N90 | 3505 | 212 | 434 | 656 | Cross-reactive Flu |
| N90 | 2115 | 213 | 435 | 657 | Cross-reactive Flu |
| N90 | 2724 | 214 | 436 | 658 | Cross-reactive Flu |
| N90 | 3436 | 215 | 437 | 659 | Cross-reactive Flu |
| N90 | 2678 | 216 | 438 | 660 | Cross-reactive Flu |
| N90 | 645 | 217 | 439 | 661 | Cross-reactive Flu |
| N90 | 3007 | 218 | 440 | 662 | Cross-reactive Flu |
| N90 | 2539 | 219 | 441 | 663 | Cross-reactive Flu |
| N90 | 1900 | 220 | 442 | 664 | Cross-reactive Flu |
| N90 | 1499 | 221 | 443 | 665 | Cross-reactive Flu |
| N90 | 1367 | 222 | 444 | 666 | Cross-reactive Flu |

TABLE 2
Amino acid sequences for heavy and light chains and the CDRs thereof.

| | SEQ ID | | | | SEQ ID | | | | |
|---|---|---|---|---|---|---|---|---|---|
| | NO for | | | | NO for | | | | |
| | Heavy | SEQ ID | SEQ ID | SEQ ID | Light | SEQ ID | SEQ ID | SEQ ID | |
| mAb | chain | NO for | NO for | NO for | chain | NO for | NO for | NO for | |
| name | aa | CDRH1 | CDRH2 | CDRH3 | aa | CDRL1 | CDRL2 | CDRL3 | Specificity |
| 2723-4872 | 667 | 734 | 761 | 796 | 844 | 852 | 878 | 903 | HIV |
| 3602-2648 | 668 | 721 | 746 | 784 | 830 | 863 | 888 | 897 | HIV |
| 3602-3278 | 668 | 721 | 746 | 784 | 830 | 863 | 888 | 897 | HIV |
| 3602-520 | 668 | 721 | 746 | 784 | 830 | 863 | 888 | 897 | HIV |
| 2723-432 | 669 | 720 | 766 | 774 | 810 | 864 | 882 | 899 | HIV |
| 3602-1483 | 670 | 714 | 744 | 794 | 829 | 862 | 891 | 897 | HIV |
| 3602-1075 | 671 | 719 | 745 | 776 | 815 | 872 | 889 | 898 | HIV |
| 3602-2137 | 672 | 719 | 745 | 776 | 816 | 869 | 889 | 898 | HIV |
| 3602-2199 | 673 | 719 | 745 | 776 | 814 | 866 | 889 | 901 | HIV |
| 3602-3420 | 674 | 722 | 742 | 793 | 831 | 867 | 889 | 894 | HIV |
| 3602-1337 | 675 | 717 | 743 | 793 | 812 | 865 | 889 | 896 | HIV |
| 3602-1494 | 675 | 717 | 743 | 793 | 812 | 865 | 889 | 896 | HIV |
| 3602-1735 | 675 | 717 | 743 | 793 | 812 | 865 | 889 | 896 | HIV |
| 3602-2848 | 675 | 717 | 743 | 793 | 812 | 865 | 889 | 896 | HIV |
| 3602-392 | 675 | 717 | 743 | 793 | 812 | 865 | 889 | 896 | HIV |
| 3602-964 | 675 | 717 | 743 | 793 | 812 | 865 | 889 | 896 | HIV |
| 3602-1544 | 676 | 717 | 743 | 791 | 811 | 865 | 889 | 895 | HIV |
| 3602-1841 | 676 | 715 | 743 | 791 | 811 | 865 | 889 | 895 | HIV |
| 3602-1737 | 677 | 718 | 743 | 793 | 811 | 865 | 889 | 895 | HIV |
| 3602-819 | 677 | 718 | 743 | 785 | 811 | 865 | 889 | 895 | HIV |
| 2723-3862 | 678 | 738 | 751 | 798 | 832 | 855 | 877 | 906 | HIV |
| 2723-5847 | 678 | 738 | 751 | 798 | 833 | 855 | 877 | 906 | HIV |
| 2723-483 | 679 | 736 | 747 | 783 | 827 | 848 | 881 | 908 | HIV |
| 2723-7033 | 680 | 736 | 747 | 783 | 828 | 847 | 880 | 908 | HIV |
| 2723-6307 | 681 | 736 | 747 | 783 | 828 | 847 | 880 | 908 | HIV |
| 2723-4196 | 682 | 736 | 747 | 782 | 825 | 848 | 880 | 908 | HIV |
| 2723-1241 | 683 | 736 | 747 | 783 | 826 | 848 | 881 | 908 | HIV |
| 2723-4559 | 684 | 735 | 748 | 800 | 822 | 850 | 880 | 904 | HIV |
| 3602-870 | 685 | 713 | 749 | 773 | 813 | 851 | 879 | 893 | HIV |
| 3602-1707 | 686 | 723 | 752 | 768 | 809 | 868 | 889 | 900 | flu |
| 2723-2304 | 687 | 725 | 762 | 778 | 818 | 859 | 885 | 913 | HIV |
| 2723-422 | 688 | 726 | 763 | 780 | 817 | 859 | 885 | 913 | HIV |
| 2723-3415 | 689 | 739 | 753 | 777 | 819 | 860 | 878 | 909 | flu |
| 2723-2120 | 690 | 727 | 741 | 775 | 834 | 873 | 884 | 916 | HIV |
| 2723-2121 | 691 | 728 | 767 | 779 | 821 | 871 | 883 | 912 | HIV |
| 2723-1952 | 692 | 740 | 756 | 781 | 808 | 870 | 887 | 892 | HIV |
| 2723-3196 | 693 | 716 | 764 | 790 | 807 | 857 | 879 | 914 | HIV |
| 2723-2859 | 694 | 724 | 757 | 799 | 820 | 861 | 883 | 910 | flu |
| 2723-5469 | 695 | 730 | 758 | 787 | 839 | 876 | 890 | 911 | HIV |
| 2723-293 | 696 | 731 | 760 | 788 | 835 | 874 | 890 | 911 | HIV |
| 2723-4186 | 696 | 731 | 760 | 788 | 840 | 858 | 890 | 911 | HIV |
| 2723-2540 | 697 | 733 | 765 | 786 | 838 | 876 | 890 | 911 | HIV |
| 2723-3244 | 698 | 732 | 758 | 788 | 837 | 875 | 890 | 911 | HIV |
| 2723-6220 | 699 | 732 | 758 | 789 | 837 | 875 | 890 | 911 | HIV |
| 2723-5655 | 700 | 732 | 758 | 788 | 837 | 875 | 890 | 911 | HIV |
| 2723-6684 | 701 | 731 | 760 | 788 | 836 | 874 | 890 | 911 | HIV |
| 2723-2624 | 702 | 729 | 750 | 792 | 841 | 853 | 886 | 915 | HIV |
| 2723-5479 | 703 | 729 | 750 | 792 | 842 | 853 | 886 | 915 | HIV |
| 2723-3069 | 704 | 737 | 759 | 801 | 824 | 849 | 880 | 905 | HIV |
| 2723-4975 | 704 | 737 | 759 | 801 | 823 | 846 | 880 | 905 | HIV |
| 2723-6609 | 704 | 737 | 759 | 801 | 823 | 846 | 880 | 905 | HIV |
| 2723-3055 | 705 | 729 | 761 | 795 | 843 | 853 | 886 | 902 | HIV |
| 2723-3131 | 706 | 712 | 754 | 772 | 806 | 856 | 883 | 907 | HIV |
| 2723-4886 | 707 | 712 | 754 | 769 | 802 | 856 | 883 | 907 | HIV |
| 2723-4509 | 708 | 712 | 755 | 770 | 804 | 856 | 883 | 907 | HIV |
| 2723-1879 | 709 | 712 | 755 | 771 | 803 | 856 | 883 | 907 | HIV |
| 2723-229 | 710 | 712 | 755 | 770 | 805 | 856 | 883 | 907 | HIV |
| 2723-6245 | 711 | 734 | 761 | 797 | 845 | 854 | 885 | 903 | HIV |

TABLE 3
Sequences in FIG. 2.

| SEQ ID | | SEQ ID | |
|---|---|---|---|
| NO | CDRH3 | NO | CDRL3 |
| 770 | AMRDYCRDDNCNKWDLRH | 907 | QHRET |
| 771 | AMRDYCRDDNCNRWDLRH | 907 | QHRET |
| 917 | AMRDYCRDDSCNIWDLRH | 907 | QHRET |
| 918 | AMRDYCRDDNCNIWDLRH | 907 | QHRET |
| 919 | VRTAYCERDPCKGWVFPH | 906 | QFLEN |
| 920 | VRRFVCDHCSDYTFGH | 904 | QDQEF |
| 921 | VRRGHCDHCYEWTLQH | 905 | QDRQS |
| 922 | VRRGSCDYCGDFPWQY | 908 | QQFEF |
| 923 | VRRGSCGYCGDFPWQY | 908 | QQFEF |
| 924 | VRGSSCCGGRRHCNGADCFNWDFQY | 903 | QCLEA |
| 925 | VRGRSCCGGRRHCNGADCFNWDFQY | 903 | QCLEA |
| 926 | VRGKSCCGGRRYCNGADCFNWDFEH | 915 | QSFEG |
| 927 | VRGRSCCDGRRYCNGADCFNWDFEH | 902 | QCFEG |
| 928 | TRGKYCTARDYYNWDFEH | 911 | QQYEF |
| 929 | TRGKYCTARDYYNWDFEY | 911 | QQYEF |
| 930 | TRGKNCDDNWDFEH | 911 | QQYEF |
| 931 | TRGKNCNYNWDFEH | 911 | QQYEF |

TABLE 4
Additional sequences in FIG. 2.

| SEQ | | SEQ | |
|---|---|---|---|
| ID | | ID | VJ |
| NO | VDJ Junction | NO | junction |
| 939 | ARHRADYDFWNGNNLRGYFDP | 912 | QQYGSSPTT |
| 940 | ARHRANYDFWGGSNLRGYFDP | 913 | QQYGTSPTT |
| 941 | ARHRADYDFWGGSNLRGYFDP | 913 | QQYGTSPTT |
| 942 | ARDEVLRGSASWFLGPNEVRHYGMDV | 899 | MQSLQLRS |
| 943 | VGRQKYISGNVGDFDF | 914 | QQYTNLPPALN |
| 944 | ATGRIAASGFYFQH | 892 | HHYNSFSHT |
| 775 | AREHTMIFGVAEGFWFDP | 916 | SSRDTDDISVI |
| 945 | VTMSGYHVSNTYLDA | 910 | QQYANSPLT |
| 946 | ARGRVYSDY | 909 | QQSGTSPPWT |

TABLE 5
Sequences in FIG. 3.

| SEQ ID NO | CDRH3 | SEQ ID NO | CDRL3 |
|---|---|---|---|
| 932 | VRGPSSGWWYHEYSGLDV | 897 | MQARQTPRLS |
| 933 | IRGPESGWFYHYYFGLGV | 897 | MQARQTPRLS |
| 934 | ARGPSSGWHLHYYFGMGL | 937 | MQSLETPRLS |
| 934 | ARGPSSGWHLHYYFGMGL | 938 | MQSLQTPRLS |
| 935 | VRGPSSGWHLHYYFGMDL | 894 | MEALQTPRLT |
| 935 | VRGPSSGWHLHYYFGMDL | 896 | METLQTPRLT |
| 935 | VRGPSSGWHLHYYFGMDL | 895 | MESLQTPRLT |
| 936 | VRGASSGWHLHYYFGMDL | 895 | MESLQTPRLT |

TABLE 6
Additional sequences in FIG. 3.

| SEQ ID | | SEQ ID | |
|---|---|---|---|
| NO | VDJ Junction | NO | VJ junction |
| 947 | ARDAGERGLRGYSVGFFDS | 893 | HQYGTTPYT |
| 948 | AKVVAGGQLRYFDWQEGHYYGMDV | 900 | MQSLQTPHS |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method for simultaneous detection of an antigen and an antibody that specifically binds said antigen, comprising:
labeling a plurality of antigens with unique antigen barcodes;
providing a plurality of barcode-labeled antigens to a population of B-cells;
allowing the plurality of barcode-labeled antigens to bind to the population of B-cells;
washing unbound antigens from the population of B-cells;
separating the B-cells into single cell emulsions;
introducing into each single cell emulsion a unique cell barcode-labeled bead;
preparing a single cell cDNA library from the single cell emulsions;
performing PCR amplification reactions to produce a plurality of amplicons, wherein the amplicons comprise: 1) the cell barcode and the antigen barcode, 2) the cell barcode and an antibody sequence, and 3) a unique molecular identifier (UMI);
sequencing the plurality of amplicons;
removing a sequence lacking the cell barcode, the UMI, or the antigen barcode;
aligning the antibody sequence to a reference library of immunoglobulin V, D, J and C sequences;
constructing a UMI count matrix comprising the cell barcode, the antigen barcode, and the antibody sequence;
determining a LIBRA-seq score; and
determining that the antibody specifically binds an antigen if the LIBRA-seq score of the antibody for the antigen is increased in comparison to a control sample.

2. The method of claim 1, wherein the barcode-labeled antigens are labeled with a first barcode comprising a DNA sequence or an RNA sequence.

3. The method of claim 1, wherein the cell barcode-labeled beads are labeled with a second barcode comprising a DNA sequence or an RNA sequence.

4. The method of claim 1, wherein the antibody sequence comprises an immunoglobulin heavy chain (VDJ) sequence, or an immunoglobulin light chain (VJ) sequence.

5. The method of claim 1, wherein the barcode-labeled antigens comprise an antigen from a pathogen or an animal.

6. The method of claim 5, wherein the antigen from a pathogen comprises an antigen from a virus.

7. The method of claim 6, wherein the antigen from a virus comprises an antigen from human immunodeficiency virus (HIV), an antigen from influenza virus, or an antigen from respiratory syncytial virus (RSV).

8. The method of claim 1, further comprising determining a level of somatic hypermutation of the antibody specifically binding to the antigen.

9. The method of claim 1, further comprising determining a length of a complementarity-determining region (CDR) of the antibody specifically binding to the antigen.

10. The method of claim 1, further comprising determining a motif of a CDR of the antibody specifically binding to the antigen.

11. The method of claim 9, wherein the CDR is selected from the group consisting of CDRH1, CDRH2, CDRH3, CDRL1, CDRL2, and CDRL3.

12. A method of determining a broadly neutralizing antibody to a pathogen, said method comprising:
labeling a plurality of antigens derived from the pathogen with unique antigen barcodes;
providing a plurality of barcode-labeled antigens to a population of B-cells;
allowing the plurality of barcode-labeled antigens to bind to the population of B-cells;
washing unbound antigens from the population of B-cells;
separating the B-cells into single cell emulsions;
introducing into each single cell emulsion a unique cell barcode-labeled bead;
preparing a single cell cDNA library from the single cell emulsions;
performing PCR amplification reactions to produce a plurality of amplicons, wherein the amplicons comprise: 1) the cell barcode and the antigen barcode, 2) the cell barcode and an antibody sequence, and 3) a unique molecular identifier (UMI);
sequencing the plurality of amplicons;
removing a sequence lacking a cell barcode, unique molecular identifier (UMI), or an antigen barcode;
aligning the antibody sequence to a reference library of immunoglobulin V, D, J and C sequences;
constructing a UMI count matrix comprising the cell barcode, the antigen barcode, and the antibody sequence;
determining a LIBRA-seq score; and
determining that the antibody is a broadly neutralizing antibody if the LIBRA-seq scores of the antibody for two or more antigens are increased in comparison to a control.

13. The method of claim 12, wherein the barcode-labeled antigens are labeled with a first barcode comprising a DNA sequence or an RNA sequence.

14. The method of claim 12, wherein the cell barcode-labeled beads are labeled with a second barcode comprising a DNA sequence or an RNA sequence.

15. The method of claim 12, wherein the antibody sequence comprises an immunoglobulin heavy chain (VDJ) sequence, or an immunoglobulin light chain (VJ) sequence.

16. The method of claim 12, wherein the barcode-labeled antigens comprise an antigen from a pathogen or an animal.

17. The method of claim 16, wherein the antigen from a pathogen comprises an antigen from a virus.

18. The method of claim 17, wherein the antigen from a virus comprises an antigen from human immunodeficiency virus (HIV), an antigen from influenza virus, or an antigen from respiratory syncytial virus (RSV).

19. The method of claim 12, further comprising determining a level of somatic hypermutation of the antibody specifically binding to the antigen.

20. The method of claim 12, further comprising determining a length of a complementarity-determining region (CDR) of the antibody specifically binding to the antigen.

21. The method of claim 12, further comprising determining a motif of a CDR of the antibody specifically binding to the antigen.

22. The method of claim 20, wherein the CDR is selected from the group consisting of CDRH1, CDRH2, CDRH3, CDRL1, CDRL2, and CDRL3.
